(19) United States
(12) Patent Application Publication
(10) Pub. No.: US 2015/0033408 A1
(43) Pub. Date: Jan. 29, 2015

(54) POLYPEPTIDE INVOLVED IN MORPHOGENESIS AND/OR ENVIRONMENTAL STRESS RESISTANCE OF PLANT

(71) Applicant: RIKEN, Wako-shi, Saitama (JP)

(72) Inventors: Kousuke Hanada, Yokohama-shi (JP); Mieko Higuchi, Yokohama-shi (JP); Takeshi Yoshizumi, Wako-shi (JP); Kentaro Nakaminami, Yokohama-shi (JP); Masanori Okamoto, Yokohama-shi (JP); Motoaki Seki, Yokohama-shi (JP); Minami Matsui, Yokohama-shi (JP)

(73) Assignee: RIKEN, Wako-shi, Saitama (JP)

(21) Appl. No.: 14/385,068

(22) PCT Filed: Mar. 15, 2013
(86) PCT No.: PCT/JP2013/058458

§ 371 Date: Sep. 12, 2014

(30) Foreign Application Priority Data
Mar. 15, 2012 (JP) 2012-059415

(51) Int. Cl. C12N 15/82; C07K 14/415

(52) U.S. Cl. CPC C12N 15/8271; C07K 14/415
USPC 800/278; 530/370; 530/324; 504/139; 504/138; 800/298

(57) ABSTRACT

Provided is a plant having a desired phenotype and/or desired stress resistance. Provided is a transgenic plant overexpressing a polypeptide that has the activity to control plant morphology or the activity to enhance the environmental stress resistance of plants, wherein the polypeptide(s) is one polypeptide or are a plurality of polypeptides selected from among polypeptides comprising the amino acid sequences represented by SEQ ID NOS: 1-317.

### TECHNICAL FIELD

The present invention relates to a plant having a desired phenotype and/or desired environmental stress resistance, and a method for imparting a desired phenotype and/or desired environmental stress resistance to a plant.

### BACKGROUND ART

Plant hormones have functions to accelerate or suppress plant growth, and to increase the size of fruits, for example. Plant hormones are conventionally utilized widely for cultivation of crops.

In recent years, it has been revealed and noted that some low-molecular-weight peptides to be encoded by relatively small genes expressed in plants exhibit hormone-like bioactivity and play important roles in plant development and morphogenesis.

However, research on the functions of such plant low-molecular-weight peptides has not been progressed well. Development of a useful low-molecular-weight peptide and new farming techniques using the same has been desired in the art.

### SUMMARY OF THE INVENTION

Objectives of the present invention are to provide a plant having a desired phenotype and/or desired environmental stress resistance, and to provide a method for imparting a desired phenotype and/or desired stress resistance to a plant.

As a result of intensive studies to attain the above objectives, the present inventors have discovered 75 types of polypeptides involved in control of plant morphology and plant environmental stress resistance from plants such as Arabidopsis (Arabidopsis thaliana) and thus have completed the present invention.

Specifically, the present invention is as follows.

[1] A method for producing a transgenic plant, comprising introducing a gene encoding a polypeptide that has the activity to control plant morphology or a polypeptide that has the activity to enhance the environmental stress resistance of plants into a host plant and then causing the overexpression thereof, wherein the polypeptide(s) is one polypeptide or are a plurality of polypeptides selected from the following (a) to (d):
(a) a polypeptide comprising any one of the amino acid sequences represented by SEQ ID NOS: 1-317;
(b) a polypeptide comprising an amino acid sequence that has a deletion, a substitution, an addition or an insertion of 1 or several amino acids with respect to any one of the amino acid sequences represented by SEQ ID NOS: 1-317, and having the activity to control plant morphology or the activity to enhance the environmental stress resistance of plants;
(c) a polypeptide comprising an amino acid sequence that has 60% or more identity with any one of the amino acid sequences represented by SEQ ID NOS: 1-317, and having the activity to control plant morphology or the activity to enhance the environmental stress resistance of plants; and
(d) a polypeptide, which is obtained by the expression of a gene encoding any one of the polypeptides (a) to (c) in a plant cell and has the activity to control plant morphology or the activity to enhance the environmental stress resistance of plants.
[2] The method of [1], wherein the polypeptide(s) has the activity to control plant morphology and is one polypeptide or are a plurality of polypeptides selected from the following (e) to (h):
(e) a polypeptide comprising any one of the amino acid sequences represented by SEQ ID NOS:1-69;
(f) a polypeptide comprising an amino acid sequence that has a deletion, a substitution, an addition, or an insertion of 1 or several amino acids with respect to any one of the amino acid sequences represented by SEQ ID NOS: 1-69, and having the activity to control plant morphology;
(g) a polypeptide comprising an amino acid sequence that has 60% or more identity with any one of the amino acid sequences represented by SEQ ID NOS: 1-69, and having the activity to control plant morphology; and
(h) a polypeptide, which is obtained by the expression of a gene encoding any one of the polypeptides (e) to (g) in a plant cell, and has the activity to control plant morphology.
[3] The method of [1], wherein the polypeptide(s) has the activity to enhance the environmental stress resistance of plants and is one polypeptide or are a plurality of polypeptides selected from the following (i) to (l):
(i) a polypeptide comprising any one of the amino acid sequences represented by SEQ ID NOS: 15, 21, and 70-75;
(j) a polypeptide comprising an amino acid sequence that has a deletion, a substitution, an addition or an insertion of 1 or several amino acids with respect to any one of the amino acid sequences represented by SEQ ID NOS: 15, 21 and 70-75, and having the activity to enhance the environmental stress resistance of plants;
(k) a polypeptide comprising an amino acid sequence that has 60% or more identity with any one of the amino acid sequences represented by SEQ ID NOS: 15, 21 and 70-75, and having the activity to enhance the environmental stress resistance of plants;
(l) a polypeptide, which is obtained by the expression of a gene encoding any one of the polypeptides (i) to (k) in a plant cell and has the activity to enhance the environmental stress resistance of plants.
[4] A transgenic plant overexpressing a polypeptide that has the activity to control plant morphology or a polypeptide that has the activity to enhance the environmental stress resistance of plants, wherein the polypeptide(s) is one polypeptide or are a plurality of polypeptides selected from the following (a) to (d):
(a) a polypeptide comprising any one of the amino acid sequences represented by SEQ ID NOS: 1-317;
(b) a polypeptide comprising an amino acid sequence that has a deletion, a substitution, an addition or an insertion of 1 or several amino acids with respect to any one of the amino acid sequences represented by SEQ ID NOS: 1-317, and having the activity to control plant morphology or the activity to enhance the environmental stress resistance of plants;
(c) a polypeptide comprising an amino acid sequence that has 60% or more identity with any one of the amino acid sequences represented by SEQ ID NOS: 1-317, and having the activity to control plant morphology or the activity to enhance the environmental stress resistance of plants;
(d) a polypeptide, which is obtained by the expression of a gene encoding any one of the polypeptides (a) to (c) in a plant cell and has the activity to control plant morphology or the activity to enhance the environmental stress resistance of plants.
[5] The transgenic plant of [4], wherein the polypeptide(s) has the activity to control plant morphology and is one polypeptide or are a plurality of polypeptides selected from the following (e) to (h):
(e) a polypeptide comprising any one of the amino acid sequences represented by SEQ ID NOS: 1-69;
(f) a polypeptide comprising an amino acid sequence that has a deletion, a substitution, an addition or an insertion of 1 or several amino acids with respect to any one of the amino acid sequences represented by SEQ ID NOS: 1-69, and having the activity to control plant morphology;
(g) a polypeptide comprising an amino acid sequence that has 60% or more identity with any one of the amino acid sequences represented by SEQ ID NOS: 1-69, and having the activity to control plant morphology;
(h) a polypeptide, which is obtained by the expression of a gene encoding any one of the polypeptides (e) to (g) in a plant cell and has the activity to control plant morphology.
[6] The transgenic plant of [4], wherein the polypeptide(s) has the activity to enhance the environmental stress resistance of plants and is one polypeptide or are a plurality of polypeptides selected from the following (i) to (l):
(i) a polypeptide comprising any one of the amino acid sequences represented by SEQ ID NOS: 15, 21 and 70-75;
(j) a polypeptide comprising an amino acid sequence that has a deletion, a substitution, an addition or an insertion of 1 or several amino acids with respect to any one of the amino acid sequences represented by SEQ ID NOS: 15, 21 and 70-75, and having the activity to enhance the environmental stress resistance of plants;
(k) a polypeptide comprising an amino acid sequence that has 60% or more identity with any one of the amino acid sequences represented by SEQ ID NOS: 15, 21 and 70-75, and having the activity to enhance the environmental stress resistance of plants;
(l) a polypeptide, which is obtained by the expression of a gene encoding any one of the polypeptides (i) to (k) in a plant cell and has the activity to enhance the environmental stress resistance of plants.
[7] An agricultural composition, containing a polypeptide that has the activity to control plant morphology or a polypeptide that has the activity to enhance the environmental stress resistance of plants, wherein the polypeptide(s) is one polypeptide or are a plurality of polypeptides selected from the following (a) to (d):
(a) a polypeptide comprising any one of the amino acid sequences represented by SEQ ID NOS: 1-317;
(b) a polypeptide comprising an amino acid sequence that has a deletion, a substitution, an addition or an insertion of 1 or several amino acids with respect to any one of the amino acid sequences represented by SEQ ID NOS: 1-317, and having the activity to control plant morphology or the activity to enhance the environmental stress resistance of plants;
(c) a polypeptide comprising an amino acid sequence that has 60% or more identity with any one of the amino acid sequences represented by SEQ ID NOS: 1-317, and having the activity to control plant morphology or the activity to enhance the environmental stress resistance of plants;
(d) a polypeptide, which is obtained by the expression of a gene encoding any one of the polypeptides (a) to (c) in a plant cell and has the activity to control plant morphology or the activity to enhance the environmental stress resistance of plants.
[8] The composition of [7], wherein the polypeptide(s) has the activity to control plant morphology and is one polypeptide or are a plurality of polypeptides selected from the following (e) to (h):
(e) a polypeptide comprising any one of the amino acid sequences represented by SEQ ID NOS: 1-69;
(f) a polypeptide comprising an amino acid sequence that has a deletion, a substitution, an addition or an insertion of 1 or several amino acids with respect to any one of the amino acid sequences represented by SEQ ID NOS: 1-69, and having the activity to control plant morphology;
(g) a polypeptide comprising an amino acid sequence that has 60% or more identity with any one of the amino acid sequences represented by SEQ ID NOS: 1-69, and having the activity to control plant morphology;
(h) a polypeptide, which is obtained by the expression of a gene encoding any one of the polypeptides (e) to (g) in a plant cell and has the activity to control plant morphology.
[9] The composition of [7], wherein the polypeptide(s) has the activity to enhance the stress resistance of plants and is one polypeptide or are a plurality of polypeptides selected from the following (i) to (l):
(i) a polypeptide comprising any one of the amino acid sequences represented by SEQ ID NOS: 15, 21 and 70-75;
(j) a polypeptide comprising an amino acid sequence that has a deletion, a substitution, an addition or an insertion of 1 or several amino acids with respect to any one of the amino acid sequences represented by SEQ ID NOS: 15, 21 and 70-75, and having the activity to enhance the environmental stress resistance of plants;
(k) a polypeptide comprising an amino acid sequence that has 60% or more identity with any one of the amino acid sequences represented by SEQ ID NOS: 15, 21 and 70-75, and having the activity to enhance the environmental stress resistance of plants;
(l) a polypeptide, which is obtained by the expression of a gene encoding any one of the polypeptides (i) to (k) in a plant cell and has the activity to enhance the environmental stress resistance of plants.
[10] A method for growing plants, comprising applying a polypeptide that has the activity to control plant morphology or a polypeptide that has the activity to enhance the stress resistance of plants to a plant, wherein the polypeptide(s) is one polypeptide or are a plurality of polypeptides selected from the following (a) to (d):
(a) a polypeptide comprising any one of the amino acid sequences represented by SEQ ID NOS: 1-317;
(b) a polypeptide comprising an amino acid sequence that has a deletion, a substitution, an addition or an insertion of 1 or several amino acids with respect to any one of the amino acid sequences represented by SEQ ID NOS: 1-317, and having the activity to control plant morphology or the activity to enhance the environmental stress resistance of plants;
(c) a polypeptide comprising an amino acid sequence that has 60% or more identity with any one of the amino acid sequences represented by SEQ ID NOS: 1-317, and having the activity to control plant morphology or the activity to enhance the environmental stress resistance of plants;
(d) a polypeptide, which is obtained by the expression of a gene encoding any one of the polypeptides (a) to (c) in a plant cell and has the activity to control plant morphology or the activity to enhance the environmental stress resistance of plants.
[11] The method of [10], wherein the polypeptide(s) has the activity to control plant morphology and is one polypeptide or are a plurality of polypeptides selected from the following (e) to (h):
(e) a polypeptide comprising any one of the amino acid sequences represented by SEQ ID NOS: 1-69;
(f) a polypeptide comprising an amino acid sequence that has a deletion, a substitution, an addition or an insertion of 1 or several amino acids with respect to any one of the amino acid sequences represented by SEQ ID NOS: 1-69, and having the activity to control plant morphology;
(g) a polypeptide comprising an amino acid sequence that has 60% or more identity with any one of the amino acid sequences represented by SEQ ID NOS: 1-69, and having the activity to control plant morphology;
(h) a polypeptide, which is obtained by the expression of a gene encoding any one of the polypeptides (e) to (g) in a plant cell and has the activity to control plant morphology.
[12] The method of [10], wherein the polypeptide(s) has the activity to enhance the stress resistance of plants and is one polypeptide or are a plurality of polypeptides selected from the following (i) to (l):
(i) a polypeptide comprising any one of the amino acid sequences represented by SEQ ID NOS: 15, 21 and 70-75;
(j) a polypeptide comprising an amino acid sequence that has a deletion, a substitution, an addition or an insertion of 1 or several amino acids with respect to any one of the amino acid sequences represented by SEQ ID NOS: 15, 21 and 70-75, and having the activity to enhance the environmental stress resistance of plants;
(k) a polypeptide comprising an amino acid sequence that has 60% or more identity with any one of the amino acid sequences represented by SEQ ID NOS: 15, 21 and 70-75, and having the activity to enhance the environmental stress resistance of plants;
(l) a polypeptide, which is obtained by the expression of a gene encoding any one of the polypeptides (i) to (k) in a plant cell and has the activity to enhance the environmental stress resistance of plants.

According to the present invention, a plant having a desired phenotype and desired environmental stress resistance can be obtained.

Moreover, according to the present invention, a desired phenotype and desired environmental stress resistance can be imparted to plants.

A part or all of the content disclosed in the description and/or drawings of Japanese Patent Application No. 2012-059415, which is a priority document of the present application, is herein incorporated by reference.

### BRIEF DESCRIPTION OF THE DRAWINGS

FIG. 1-1 shows the amino acid sequences of polypeptides derived from Arabidopsis (Arabidopsis thaliana), Chinese cabbage (Brassica rapa var. glabra), soybean (Glycine max), tomato (Solanum lycopersicum), rice (Oryza sativa), and corn (Zea mays). Groups (1-75) indicate that polypeptides included in each group are in a homologous relationship, such that the polypeptides have functions equivalent to each other. Gene names listed in the columns of Arabidopsis denote names registered in The Arabidopsis Information Resource (TAIR) database.

FIG. 1-2 is a continuation of FIG. 1-1.

FIG. 1-3 is a continuation of FIG. 1-2.

FIG. 1-4 is a continuation of FIG. 1-3.

FIG. 1-5 is a continuation of FIG. 1-4.

FIG. 1-6 is a continuation of FIG. 1-5.

FIG. 1-7 is a continuation of FIG. 1-6.

FIG. 2 shows photographs showing typical phenotypes observed among various overexpression plants prepared in Example 2.

FIG. 3A shows the survival rates of flc39 (SEQ ID NO: 21)-overexpressing plants and AT33 (SEQ ID NO: 15)-overexpressing plants in a drought stress resistance test. 35S::NCED3 denotes genetically recombined Arabidopsis overexpressing the NCED3 gene and having drought resistance.

FIG. 3B shows the survival rates of AT4 (SEQ ID NO: 70)-overexpressing plants and AT35 (SEQ ID NO: 73)-overexpressing plants in a high-temperature stress resistance test.

FIG. 3C shows the survival rates of AT13 (SEQ ID NO: 71)-overexpressing plants and AT19 (SEQ ID NO: 72)-overexpressing plants in a salt stress resistance test.

FIG. 4 shows the results of GUS staining of leaves and siliques of various overexpression plants.

FIG. 5 shows a photograph showing a typical phenotype observed in an flc39 (SEQ ID NO: 21) expression-inhibited stock, in which the expression of flc39 has been inhibited by RNAi, and a graph showing days required for the flowering of the stock. RNAi 2, 4 and 5 are RNAi molecules targeting mRNA coding for flc39.

FIG. 6 shows the results of a growth test for Arabidopsis using a medium supplemented with the liquid medium of AT32-overexpressing plants.

FIG. 7 shows the results of a growth test for rice using a medium supplemented with the liquid medium components of the AT32-overexpressing plants.

### MODES FOR CARRYING OUT THE INVENTION

The present invention relates to a method for producing a transgenic plant, comprising introducing a gene encoding a polypeptide that has the activity to control plant morphology or a polypeptide that has the activity to enhance the environmental stress resistance of plants into a host plant and then causing the overexpression thereof.

The present invention further relates to a transgenic plant that overexpresses a polypeptide having the activity to control plant morphology or a polypeptide having the activity to enhance the environmental stress resistance of plants.

The polypeptides in the present invention have the activity to control plant morphology and/or the activity to enhance the environmental stress resistance of plants.

The polypeptide(s) in the present invention comprises any one of the amino acid sequences shown in SEQ ID NOS: 1-317, and preferably consists of the relevant amino acid sequence.

Examples of the polypeptide(s) in the present invention include polypeptides having a deletion, a substitution, an addition or an insertion of 1 or several or 1 or a plurality of amino acids with respect to any one of the amino acid sequences shown in SEQ ID NOS: 1-317, and having the activity to control plant morphology and/or the activity to enhance the stress resistance of plants, as in any one of the polypeptides shown in SEQ ID NOS: 1-317. Here, the range of the term “1 or a plurality of” is not particularly limited. Examples thereof include 25 or less, 20 or less, 15 or less, 10 or less, further preferably 5 or less, and particularly preferably 4 or less, or 1 or 2.

Furthermore, examples of the polypeptide(s) in the present invention include a polypeptide comprising an amino acid sequence having 30% or more, 35% or more, 40% or more, 45% or more, 50% or more, 55% or more, 60% or more, 65% or more, 70% or more, 75% or more, 80% or more, 85% or more, 90% or more, 95% or more, or 99% or more identity with any one of the amino acid sequences shown in SEQ ID NOS: 1-317, when calculated using BLAST (Basic Local Alignment Search Tool at the National Center for Biological Information (NCBI)) or the like (for example, default; that is, initially set parameters), and preferably consisting of the relevant amino acid sequence, and having the activity to control plant morphology and/or the activity to enhance the stress resistance of plants, as in any one of the polypeptides shown in SEQ ID NOS: 1-317. Examples of such a polypeptide include homologs (orthologs and paralogs) of any one of the polypeptides shown in SEQ ID NOS: 1-317. Furthermore, examples of the polypeptide(s) in the present invention include a polypeptide comprising an amino acid sequence that has the above identity with a sequence of a plurality of continuous amino acids in any one of the amino acid sequences shown in SEQ ID NOS: 1-317, and preferably consisting of the relevant amino acid sequence, and having the activity to control plant morphology and/or the activity to enhance the stress resistance of plants, as in any one of the polypeptides shown in SEQ ID NOS: 1-317. Here, the range of the “plurality of” (the number of) continuous amino acids is not particularly limited. Examples thereof include 100 or less, 90 or less, 80 or less, 70 or less, 60 or less, 50 or less, 40 or less, 30 or less, 20 or less, and 10 or less.

Examples of the polypeptides in the present invention also include peptides (functional peptides) consisting of the functional portions of the above polypeptides. Such a functional peptide can be obtained by expressing a gene encoding the above polypeptide in plant cells. Alternatively, such a functional peptide can also be specified by determining the cleavage position through the removal of secretion signals or processing. For example, secretion signals can be analyzed using a program provided on the following website (http://www.cbs.dtu.dk/services/SignalP/).

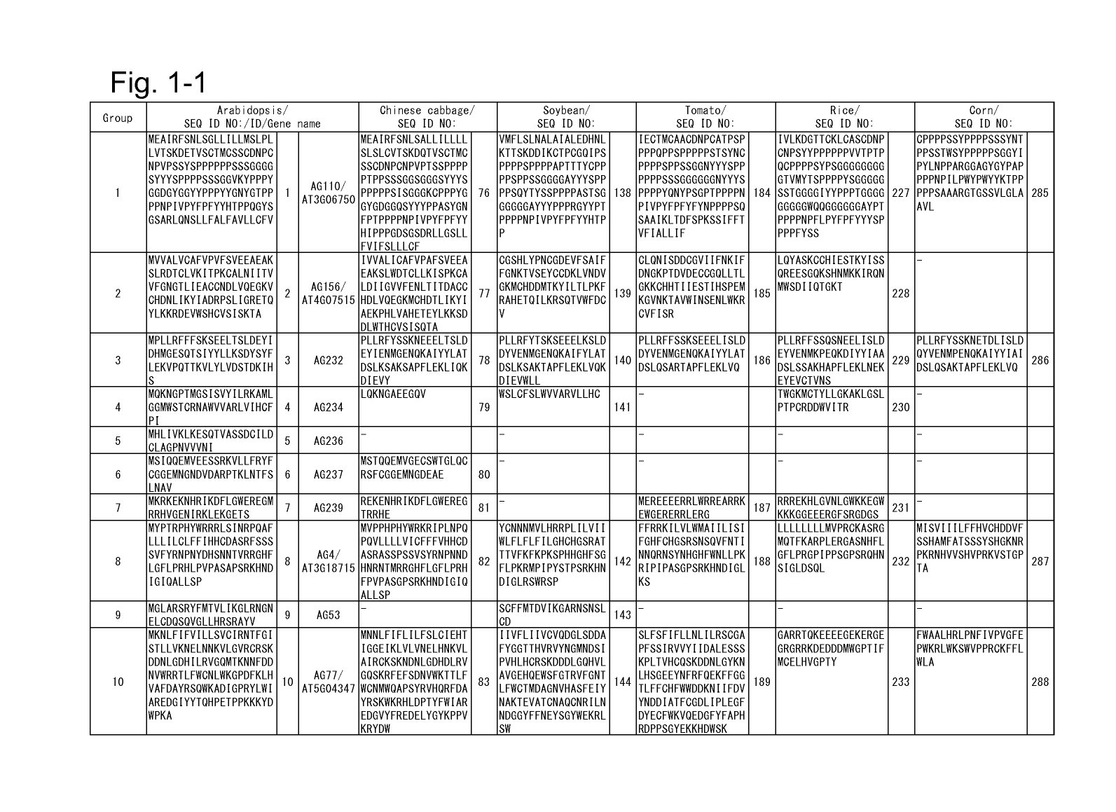

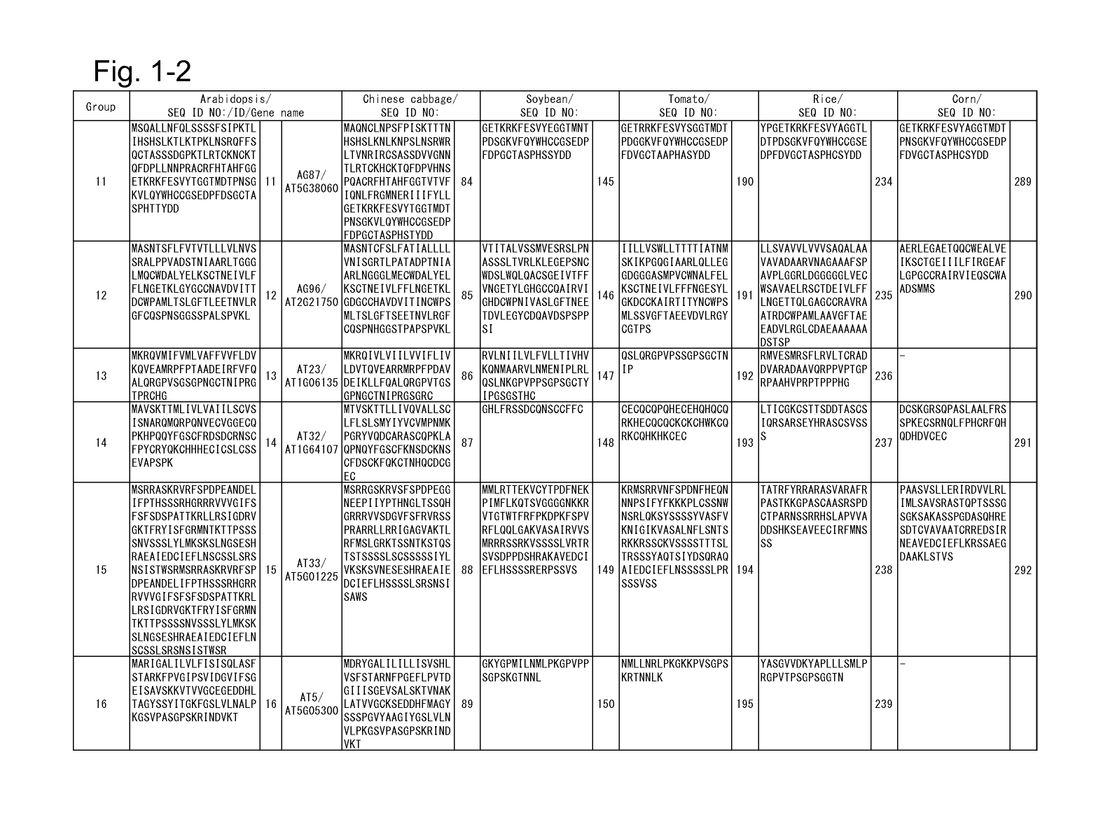

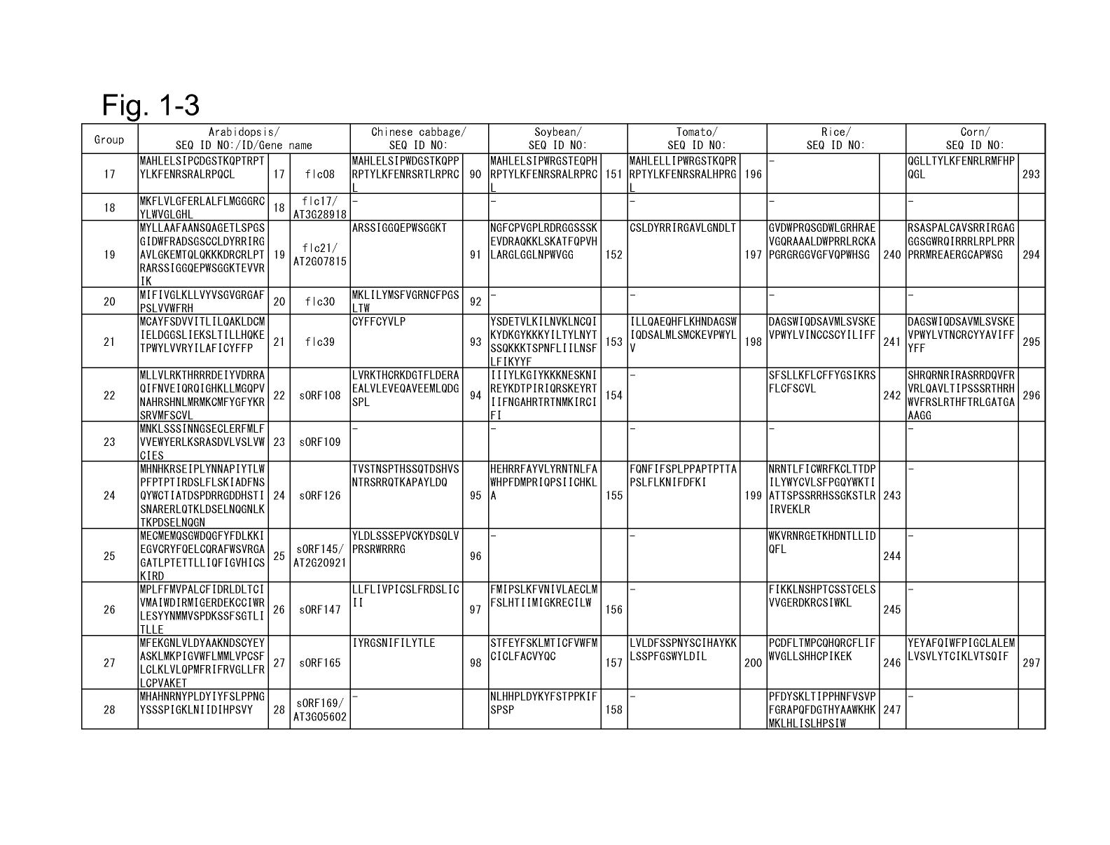

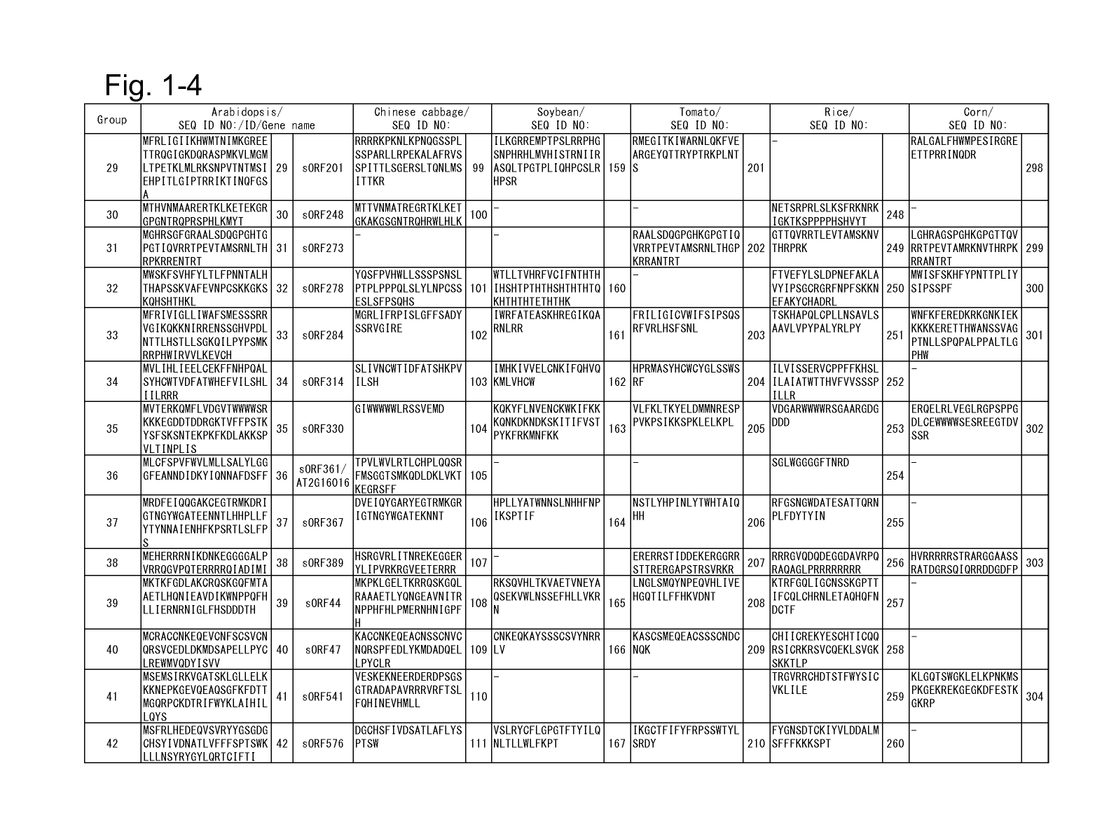

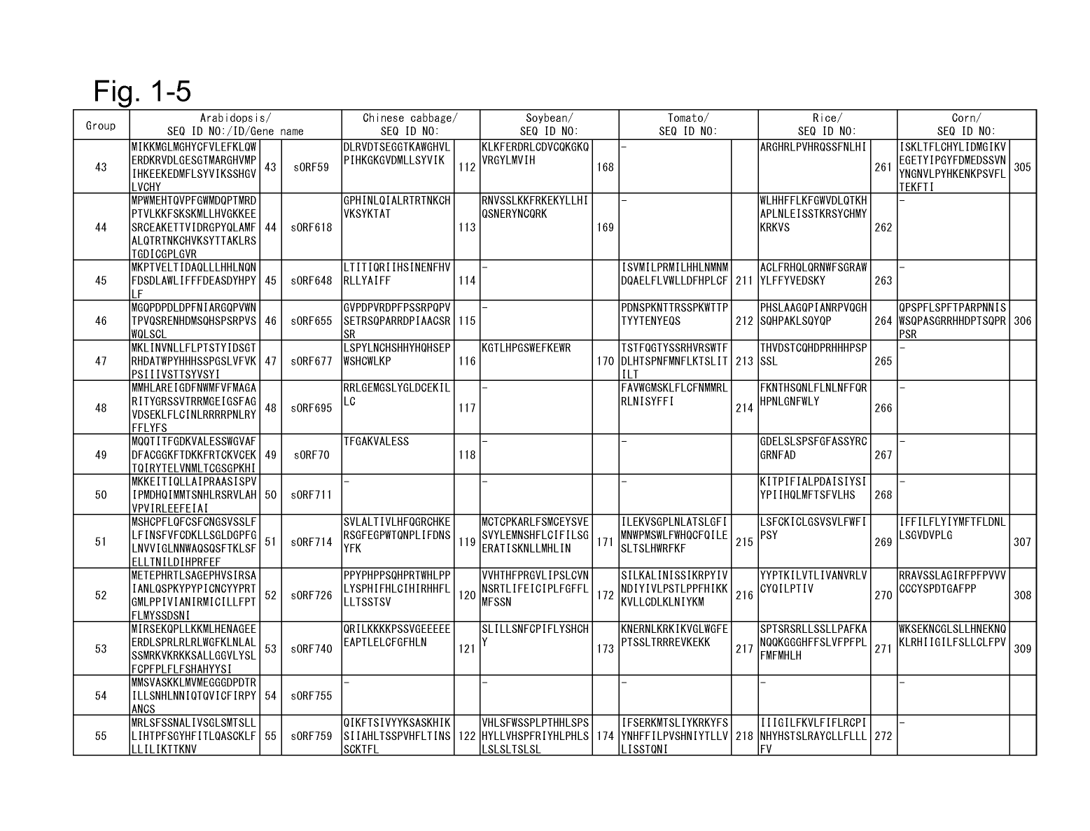

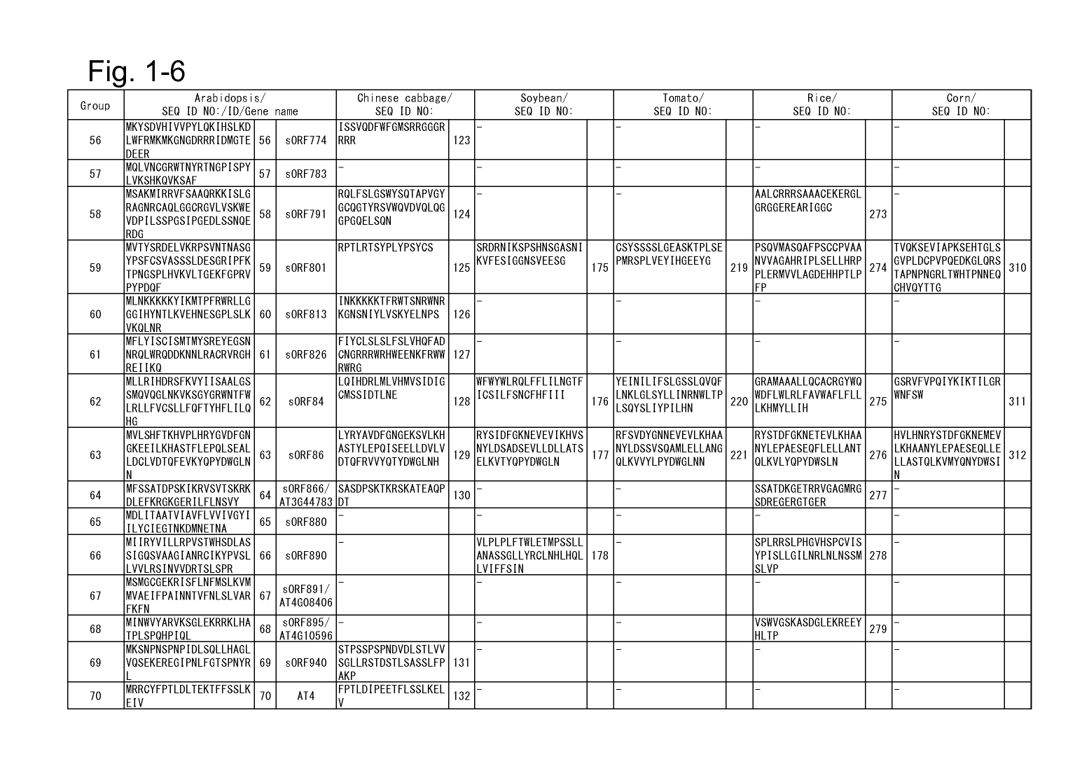

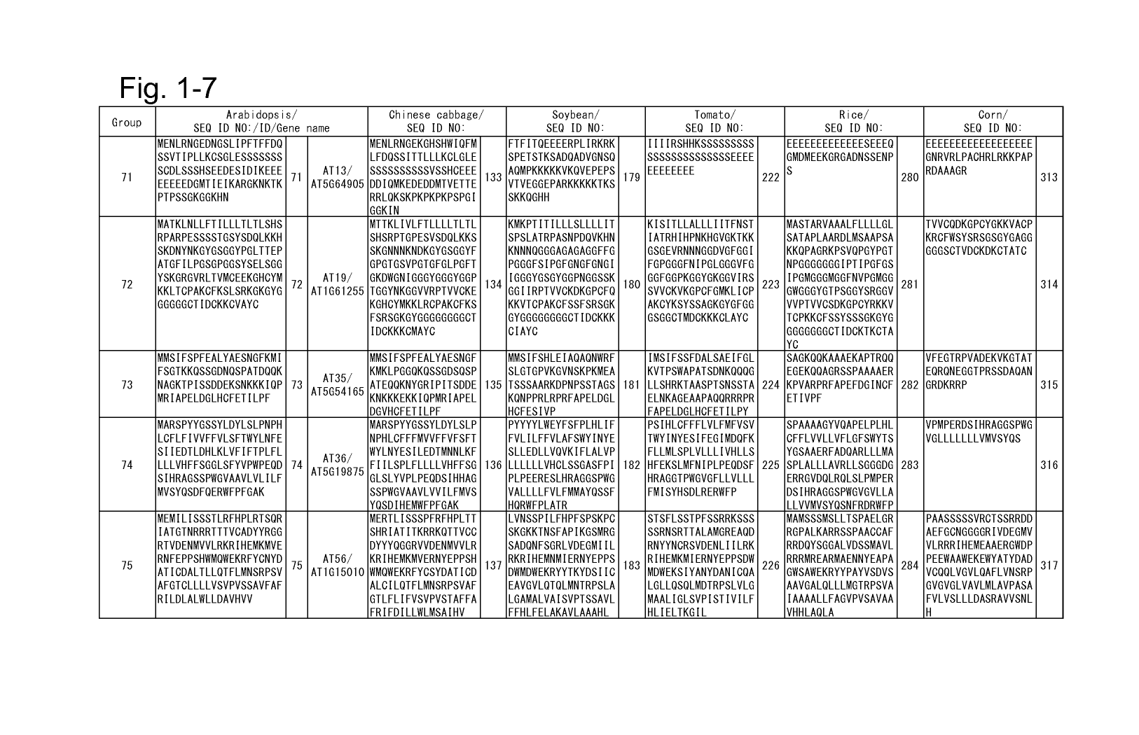

The polypeptides in the present invention can be classified as shown in FIG. 1.

Specifically, the polypeptides shown in SEQ ID NOS: 1-75 are derived from Arabidopsis (Arabidopsis thaliana). The polypeptides shown in SEQ ID NOS: 76-137 are derived from Chinese cabbage (Brassica rapa var. glabra). The polypeptides shown in SEQ ID NOS: 138-183 are derived from soybean (Glycine max). The polypeptides shown in SEQ ID NOS: 184-226 are derived from tomato (Solanum lycopersicum). The polypeptides shown in SEQ ID NOS: 227-284 are derived from rice (Oryza sativa). The polypeptides shown in SEQ ID NOS: 285-317 are derived from corn (Zea mays).

The groups (1-75) indicate that polypeptides included in each group have functions equivalent to each other (at least, they have functions equivalent to those of the polypeptides from Arabidopsis included in each group) and thus are in a homologous relationship. Specifically, the polypeptides included in group 1, which are represented by SEQ ID NOS: 1, 76, 138, 184, 227 and 285, are in the above homologous relationship. The polypeptides included in group 2, which are represented by SEQ ID NOS: 2, 77, 139, 185 and 228, are in the above homologous relationship. The polypeptides included in group 3, which are represented by SEQ ID NOS: 3, 78, 140, 186, 229 and 286, are in the above homologous relationship. The polypeptides included in group 4, which are represented by SEQ ID NOS: 4, 79, 141 and 230, are in the above homologous relationship. The polypeptides included in group 6, which are represented by SEQ ID NOS: 6 and 80, are in the above homologous relationship. The polypeptides included in group 7, which are represented by SEQ ID NOS: 7, 81, 187 and 231, are in the above homologous relationship. The polypeptides included in group 8, which are represented by SEQ ID NOS: 8, 82, 142, 188, 232 and 287, are in the above homologous relationship. The polypeptides included in group 9, which are represented by SEQ ID NOS: 9 and 143, are in the above homologous relationship. The polypeptides included in group 10, which are represented by SEQ ID NOS: 10, 83, 144, 189, 233 and 288, are in the above homologous relationship. The polypeptides included in group 11, which are represented by SEQ ID NOS: 11, 84, 145, 190, 234 and 289, are in the above homologous relationship. The polypeptides included in group 12, which are represented by SEQ ID NOS: 12, 85, 146, 191, 235 and 290, are in the above homologous relationship. The polypeptides included in group 13, which are represented by SEQ ID NOS: 13, 86, 147, 192 and 236, are in the above homologous relationship. The polypeptides included in group 14, which are represented by SEQ ID NOS: 14, 87, 148, 193, 237 and 291, are in the above homologous relationship. The polypeptides included in group 15, which are represented by SEQ ID NOS: 15, 88, 149, 194, 238 and 292, are in the above homologous relationship. The polypeptides included in group 16, which are represented by SEQ ID NOS: 16, 89, 150, 195 and 239, are in the above homologous relationship. The polypeptides included in group 17, which are represented by SEQ ID NOS: 17, 90, 151 and 293, are in the above homologous relationship. The polypeptides included in group 19, which are represented by SEQ ID NOS: 19, 91, 152, 197, 240 and 294, are in the above homologous relationship. The polypeptides included in group 20, which are represented by SEQ ID NOS: 20 and 92, are in the above homologous relationship. The polypeptides included in group 21, which are represented by SEQ ID NOS: 21, 93, 153, 198, 241 and 295, are in the above homologous relationship. The polypeptides included in group 22, which are represented by SEQ ID NOS: 22, 94, 154, 242 and 296, are in the above homologous relationship. The polypeptides included in group 24, which are represented by SEQ ID NOS: 24, 95, 155, 199 and 243, are in the above homologous relationship. The polypeptides included in group 25, which are represented by SEQ ID NOS: 25, 96 and 244, are in the above homologous relationship. The polypeptides included in group 26, which are represented by SEQ ID NOS: 26, 97, 156 and 245, are in the above homologous relationship. The polypeptides included in group 27, which are represented by SEQ ID NOS: 27, 98, 157, 200, 246 and 297, are in the above homologous relationship. The polypeptides included in group 28, which are represented by SEQ ID NOS: 28, 158 and 247, are in the above homologous relationship. The polypeptides included in group 29, which are represented by SEQ ID NOS: 29, 99, 159, 201 and 298, are in the above homologous relationship. The polypeptides included in group 30, which are represented by SEQ ID NOS: 30, 100 and 248, are in the above homologous relationship. The polypeptides included in group 31, which are represented by SEQ ID NOS: 31, 202, 249 and 299, are in the above homologous relationship. The polypeptides included in 32, which are represented by SEQ ID NOS: 32, 101, 160, 250 and 300, are in the above homologous relationship. The polypeptides included in group 33, which are represented by SEQ ID NOS: 33, 102, 161, 203, 251 and 301, are in the above homologous relationship. The polypeptides included in group 34, which are represented by SEQ ID NOS: 34, 103, 162, 204 and 252, are in the above homologous relationship. The polypeptides included in group 35, which are represented by SEQ ID NOS: 35, 104, 163, 205, 253 and 302, are in the above homologous relationship. The polypeptides included in group 36, which are represented by SEQ ID NOS: 36, 105 and 254, are in the above homologous relationship. The polypeptides included in group 37, which are represented by SEQ ID NOS: 37, 106, 164, 206 and 255, are in the above homologous relationship. The polypeptides included in group 38, which are represented by SEQ ID NOS: 38, 107, 207, 256 and 303, are in the above homologous relationship. The polypeptides included in group 39, which are represented by SEQ ID NOS: 39, 108, 165, 208 and 257, are in the above homologous relationship. The polypeptides included in group 40, which are represented by SEQ ID NOS: 40, 109, 166, 209 and 258, are in the above homologous relationship. The polypeptides included in group 41, which are represented by SEQ ID NOS: 41, 110, 259 and 304, are in the above homologous relationship. The polypeptides included in group 42, which are represented by SEQ ID NOS: 42, 111, 167, 210 and 260, are in the above homologous relationship. The polypeptides included in group 43, which are represented by SEQ ID NOS: 43, 112, 168, 261 and 305, are in the above homologous relationship. The polypeptides included in group 44, which are represented by SEQ ID NOS: 44, 113, 169 and 262, are in the above homologous relationship. The polypeptides included in group 45, which are represented by SEQ ID NOS: 45, 114, 211 and 263, are in the above homologous relationship. The polypeptides included in group 46, which are represented by SEQ ID NOS: 46, 115, 212, 264 and 306, are in the above homologous relationship. The polypeptides included in group 47, which are represented by SEQ ID NOS: 47, 116, 170, 213 and 265, are in the above homologous relationship. The polypeptides included in group 48, which are represented by SEQ ID NOS: 48, 117, 214 and 266, are in the above homologous relationship. The polypeptides included in group 49, which are represented by SEQ ID NOS: 49, 118 and 267, are in the above homologous relationship. The polypeptides included in group 50, which are represented by SEQ ID NOS: 50 and 268, are in the above homologous relationship. The polypeptides included in group 51, which are represented by SEQ ID NOS: 51, 119, 171, 215, 269 and 307, are in the above homologous relationship. The polypeptides included in group 52, which are represented by SEQ ID NOS: 52, 120, 172, 216, 270 and 308, are in the above homologous relationship. The polypeptides included in group 53, which are represented by SEQ ID NOS: 53, 121, 173, 217, 271 and 309, are in the above homologous relationship. The polypeptides included in group 55, which are represented by SEQ ID NOS: 55, 122, 174, 218 and 272, are in the above homologous relationship. The polypeptides included in group 56, which are represented by SEQ ID NOS: 56 and 123, are in the above homologous relationship. The polypeptides included in group 58, which are represented by SEQ ID NOS: 58, 124 and 273, are in the above homologous relationship. The polypeptides included in group 59, which are represented by SEQ ID NOS: 59, 125, 175, 219, 274 and 310, are in the above homologous relationship. The polypeptides included in group 60, which are represented by SEQ ID NOS: 60 and 126, are in the above homologous relationship. The polypeptides included in group 61, which are represented by SEQ ID NOS: 61 and 127, are in the above homologous relationship. The polypeptides included in group 62, which are represented by SEQ ID NOS: 62, 128, 176, 220, 275 and 311, are in the above homologous relationship. The polypeptides included in group 63, which are represented by SEQ ID NOS: 63, 129, 177, 221, 276 and 312, are in the above homologous relationship. The polypeptides included in group 64, which are represented by SEQ ID NOS: 64, 130 and 277, are in the above homologous relationship. The polypeptides included in group 66, which are represented by SEQ ID NOS: 66, 178 and 278, are in the above homologous relationship. The polypeptides included in group 68, which are represented by SEQ ID NOS: 68 and 279, are in the above homologous relationship. The polypeptides included in group 69, which are represented by SEQ ID NOS: 69 and 131, are in the above homologous relationship. The polypeptides included in group 70, which are represented by SEQ ID NOS: 70 and 132, are in the above homologous relationship. The polypeptides included in group 71, which are represented by SEQ ID NOS: 71, 133, 179, 222, 280 and 313, are in the above homologous relationship. The polypeptides included in group 72, which are represented by SEQ ID NOS: 72, 134, 180, 223, 281 and 314, are in the above homologous relationship. The polypeptides included in group 73, which are represented by SEQ ID NOS: 73, 135, 181, 224, 282 and 315, are in the above homologous relationship. The polypeptides included in group 74, which are represented by SEQ ID NOS: 74, 136, 182, 225, 283 and 316, are in the above homologous relationship. The polypeptides included in group 75, which are represented by SEQ ID NOS: 75, 137, 183, 226, 284 and 317, are in the above homologous relationship.

Examples of the term “plant” as used herein include dicotyledons and monocotyledons, as well as grasses and trees, and the term refers to one or a plurality of plants that are selected from namely useful plants such as vegetables, fruit trees, and horticultural crops belonging to (but are not limited thereto) the families Brassicaceae, Solanaceae, Gramineae, Leguminosae, Cucurbitaceae, Convolvulaceae, Liliaceae, Apiaceae, Asteraceae, Rosaceae, Rutaceae, Myrtaceae, Chenopodiaceae, Gentianaceae, Caryophyllaceae, or the like. More specific examples thereof include, but are not limited to, Arabidopsis (Arabidopsis thaliana), oilseed rape (Brassica campestris L.), cabbage (Brassica oleracea L. var. capitata L.), broccoli (Brassica oleracea L. var. botrytis L.), Chinese cabbage (Brassica campestris L. var. amplexicaulis), eggplant (Solanum melongena L.), tobacco (Nicotiana tabacum L.), tomato (Lycopersicon esculentum Mill), pimento (Capsicum annuum L. var. grossum), potato (Solanum tuberosum L.), petunia (Petunia hybrida Vilm.), rice (Oryza sativa L.), corn (Zea mays L.), wheat (Triticum aestivum L.), barley (Hordeum vulgare L.), soybean (Glycine max L.), adzuki bean (Vigna angularis Willd.), cucumber (Cucumis sativus L.), melon (Cucumis melo L.), sweet potato (Ipomoea batatas), green onion (Allium fistulosum L.), carrot (Daucus carota L.), Chrysanthemum (Chrysanthemum morifolium), lettuce (Lactuca sativa L.), rose (Rose hybrida Hort.), peach (Prunus persica), apple (Malus pumila Mill), mandarin orange (Citras unshiu), spinach (Spinacia oleracea L.), gentian (Gentiana scabra Bunge var. buergeri Maxim.), and carnation (Dianthus caryophyllus L.).

The term “activity to control plant morphology” as used herein refers to the activity to affect the size of a plant itself, the size (oversized (becoming big) or dwarfing), shape, color tone (darkening or lightening), and number (increase or decrease) of different plant organs (for example, leaves and rosettes, and stems and shoots, roots, flowers, petals, fruits, bulbs, and seeds).

The polypeptides included in groups 1-69 at least have the activity to control plant morphology.

Specifically, the polypeptides included in groups 3, 4, 5, 6, 7, 8, 9, 11, 12, 14, 17, 18, 19, 21, 23, 27, 29, 30, 31, 32, 34, 37, 39, 44, 50, 51, 52, 56, 58, 59, 60, 62, 63, 64 and 66 at least have the activity to control the size of plants or plant organs. More specifically, the polypeptides included in groups 3, 4, 5, 6, 7, 17, 18, 19, 21, 31, 37, 39, 44, 50, 51, 52, 56, 58, 59, 60 and 66 have the activity to cause oversized plants or plant organs. Further specifically, a polypeptide comprising the amino acid sequence shown in any one of SEQ ID NOS: 3, 4, 5, 6, 7, 17, 18, 19, 21, 31, 37, 39, 44, 50, 51, 52, 56, 58, 59, 60 and 66 or consisting of the relevant amino acid sequence has the activity to cause oversized plants or plant organs. Moreover, the polypeptides included in groups 8, 9, 11, 12, 14, 23, 27, 29, 30, 32, 34, 62, 63 and 64 at least have the activity to cause the dwarfing of plants or plant organs. Further specifically, a polypeptide comprising the amino acid sequence shown in any one of SEQ ID NOS: 8, 9, 11, 12, 14, 23, 27, 29, 30, 32, 34, 62, 63 and 64 or consisting of the relevant amino acid sequence has the activity to cause the dwarfing of plants or plant organs. Therefore, transformants expressing any one of or a combination of a plurality of these polypeptides and plants, to which any one of or a combination of a plurality of these polypeptides have been applied, exhibit significant changes in the size of plants or plant organs, compared with wild-type or untreated plants.

Moreover, the polypeptides included in groups 2, 20, 36, 47, 54 and 69 at least have the activity to control plant flowering. More specifically, the polypeptides included in groups 2, 20, 36, 47, 54 and 69 at least have the activity to suppress plant flowering. Further specifically, a polypeptide comprising the amino acid sequence shown in any one of SEQ ID NOS: 2, 20, 36, 47, 54 and 69 or consisting of the relevant amino acid sequence has the activity to suppress plant flowering. Therefore, transformants expressing any one of or a combination of a plurality of these polypeptides and plants, to which any one of or a combination of a plurality of these polypeptides have been applied, exhibit the significantly delayed times of plant flowering, compared with wild-type or untreated plants.

Furthermore, the polypeptides included in groups 1, 10, 13, 15, 16, 22, 24 and 28 at least have the activity to control the shape of plant leaves and rosettes. More specifically, the polypeptides included in groups 1, 10, 13, 15, 16, 22, 24 and 28 at least have the activity to cause changes in the shape of plant leaves and rosettes (for example, causing an increase or a decrease in leaf length or leaf width, causing uneven/irregular surface, and causing twist formation). Further specifically, a polypeptide comprising the amino acid sequence shown in any one of SEQ ID NOS: 1, 10, 13, 15, 16, 22, 24 and 28 or consisting of the relevant amino acid sequence has the activity to cause changes in the shape of plant leaves and rosettes. Therefore, transformants expressing any one of or a combination of a plurality of these polypeptides and plants, to which any one of or a combination of a plurality of these polypeptides have been applied, exhibit significant changes in the shape of plant leaves and rosettes, compared with wild-type or untreated plants.

Moreover, the polypeptides included in groups 25, 35, 43, 46, 49, 38 and 67 at least have the activity to control the color tone of plant leaves and rosettes. More specifically, the polypeptides included in groups 25, 35, 46 and 67 at least have the activity to darken the color tone of plant leaves and rosettes. Further specifically, a polypeptide comprising the amino acid sequence shown in any one of SEQ ID NOS: 25, 35, 46 and 67 or consisting of the relevant amino acid sequence has the activity to darken the color tone of plant leaves and rosettes. Also, the polypeptides included in groups 38, 43 and 49 at least have the activity to lighten the color tone of plant leaves and rosettes. Further specifically, a polypeptide comprising the amino acid sequence shown in any one of SEQ ID NOS: 38, 43 and 49 or consisting of the relevant amino acid sequence has the activity to lighten the color tone of plant leaves and rosettes. Therefore, transformants expressing any one of or a combination of a plurality of these polypeptides and plants, to which any one of or a combination of a plurality of these polypeptides have been applied, exhibit significant changes in the color tone of plant leaves and rosettes, compared with wild-type or untreated plants.

Further, the polypeptides included in groups 41 and 45 at least have the activity to control the shape of plant stems. More specifically, the polypeptides included in groups 41 and 45 at least have the activity to cause changes in the shape of plant stems (for example, increasing or decreasing the thickness and length, causing branching, bending, or twisting). Further specifically, a polypeptide comprising the amino acid sequence shown in either SEQ ID NO: 41 or 45, or consisting of the relevant amino acid sequence has the activity to cause changes in the shape of plant stems. Therefore, transformants expressing any one of or a combination of a plurality of these polypeptides and plants, to which any one of or a combination of a plurality of these polypeptides have been applied, exhibit significant changes in the shape of plant stems, compared with wild-type or untreated plants.

Moreover, the polypeptides included in group 48 at least have the activity to control the flower shape of plants. More specifically, the polypeptides included in group 48 at least have the activity to cause changes in the flower shape of plants (for example, causing oversized flower, causing dwarfing, increasing or decreasing the number of petals, and causing changes in color tone). Further specifically, a polypeptide comprising the amino acid sequence shown in SEQ ID NO: 48 or consisting of the relevant amino acid sequence has the activity to cause changes in the flower shape of plants. Therefore, transformants expressing any one of or a combination of a plurality of these polypeptides and plants, to which any one of or a combination of a plurality of these polypeptides have been applied, exhibit significant changes in the flower shape of plants, compared with wild-type or untreated plants.

Moreover, the polypeptides included in groups 26, 33, 40, 42, 53, 55, 57, 61, 65 and 68 at least have the activity to control the fruit formation and/or the fruit maturation of plants. More specifically, the polypeptides included in group 65 at least have the activity to cause changes in the fruit formation of plants (for example, causing uneven/irregular surface, causing oversized fruits, causing dwarfing, and causing changes in color tone). Further specifically, a polypeptide comprising the amino acid sequence shown in SEQ ID NO: 65 or consisting of the relevant amino acid sequence has the activity to cause changes in the fruit shape of plants. Also, the polypeptides included in groups 61 and 68 at least have the activity to inhibit the fruit formation of plants. More specifically, a polypeptide comprising the amino acid sequence shown in SEQ ID NO: 61 or 68 or consisting of the relevant amino acid sequence has the activity to inhibit the fruit formation of plants. Also, the polypeptides included in groups 26, 33, 40, 42, 53, 55 and 57 at least have the activity to inhibit the fruit maturation of plants. More specifically, polypeptides comprising the amino acid sequences shown in SEQ ID NOS: 26, 33, 40, 42, 53, 55 and 57 or consisting of the relevant amino acid sequences have the activity to inhibit the fruit maturation of plants. Therefore, transformants expressing any one of or a combination of a plurality of these polypeptides and plants, to which any one of or a combination of a plurality of these polypeptides have been applied, exhibit significant changes in the fruit formation and/or the fruit maturation of plants, compared with wild-type or untreated plants.

The above polypeptides may have another type of activity, in addition to the “activity to control plant morphology” as defined above.

The activity of each polypeptide can be evaluated by comparing and analyzing the phenotype of a plant body in which the expression level of the relevant polypeptide has been increased (for example, an overexpression plant described specifically below) or a plant, to which the relevant polypeptide has been applied, with the phenotype of wild-type or untreated plants.

The term “environmental stress” as used herein refers to a stress to which a plant is exposed because of the growth environment and conditions, such as stresses resulting from drought, high humidity, high temperature, low temperature, strong light, weak light, salt, air pollutants, pesticides, diseases, or the like (examples thereof are not limited thereto).

The term “activity to enhance the environmental stress resistance of plants” as used herein refers to the activity to improve the growth and/or the survival rate of plants in the presence of environmental stresses.

The polypeptides included in groups 15, 21 and 70-75 at least have the activity to enhance the environmental stress resistance of plants.

Specifically, the polypeptides included in groups 15 and 21 at least have the activity to enhance the drought stress resistance of plants. More specifically, a polypeptide comprising the amino acid sequence shown in either SEQ ID NO: 15 or 21 or consisting of the relevant amino acid sequence has the activity to enhance the drought stress resistance of plants. The term “drought stress” refers to a stress resulting from a situation when plants are exposed to continuous or temporal water depletion. Therefore, transformants expressing any one of or a combination of a plurality of these polypeptides and plants, to which any one of or a combination of a plurality of these polypeptides have been applied, can improve the growth and/or the survival rates of plants in the presence of a drought stress to a degree higher than that of wild-type or untreated plants.

Moreover, the polypeptides included in groups 70, 73 and 74 at least have the activity to enhance the high-temperature stress resistance of plants. More specifically, a polypeptide comprising the amino acid sequence shown in any one of SEQ ID NOS: 70, 73 and 74 or consisting of the relevant amino acid sequence has the activity to enhance the high-temperature stress resistance of plants. The term “high-temperature stress” refers to a stress resulting from a situation when plants are exposed to continuous or temporal conditions of 35° C. or higher for several minutes or longer, for example. Therefore, transformants expressing any one of or a combination of a plurality of these polypeptides and plants, to which any one of or a combination of a plurality of these polypeptides have been applied, can improve the growth and/or the survival rates of plants in the presence of a high-temperature stress to a degree higher than that of wild-type or untreated plants.

Moreover, the polypeptides included in groups 71, 72 and 75 at least have the activity to enhance the salt stress resistance of plants. More specifically, a polypeptide comprising the amino acid sequence shown in any one of SEQ ID NOS: 71, 72 and 75 or consisting of the relevant amino acid sequence has the activity to enhance the salt stress resistance of plants. The term “salt stress” refers to a stress resulting from damage on the physiological functions of plant bodies, when salts accumulated in soil or a medium lower the water potential of the soil and the like and thus plants become unable to absorb water, for example. Therefore, transformants expressing any one of or a combination of a plurality of these polypeptides and plants, to which any one of or a combination of a plurality of these polypeptides have been applied, can improve the growth and/or the survival rates of plants in the presence of a salt stress to a degree higher than that of wild-type or untreated plants.

The above polypeptides may have another type of activity in addition to the “activity to enhance the environmental stress resistance of plants” defined above.

The activity of each polypeptide can be evaluated by comparing and analyzing the growth or the survival rates of plant bodies in the presence of various stresses, in which the expression level of the polypeptide has been increased (for example, overexpression plants described specifically below) and plants, to which the polypeptide has been applied, with those of wild-type or untreated plants. Specifically, when a plant body, in which the expression level of the polypeptide has been increased (for example, overexpression plants specifically described below) or a plant, to which the polypeptide has been applied, significantly grow in the presence of various stresses, compared with wild-type plants or plants, to which no such polypeptide has been applied, this can be concluded that the stress resistance has been successfully imparted to the plants.

The transgenic plant of the present invention can be produced using a known transgenic technique (supervised by translation supervisors, Michio Matsuhashi et al., Molecular Biology of Watson-recombinant DNA, 2nd edition, 1994, Maruzen; Experimental Protocol of Model Plant, 3^{rd} revised edition (2005), supervised by Shimamoto, Okada and Tabata, Shujunsha). Specifically, the transgenic plant of the present invention can be prepared by ligating a gene encoding the above polypeptide under the control of a promoter, so as to incorporate it into a vector, and then introducing the vector into appropriate plant cells. A gene encoding the above polypeptide can be searched for using published database (e.g., RIKEN Arabidopsis full-length (RAFL) cDNA resources, NCBI Genbank, The Arabidopsis Information Resource (TAIR)). A gene from a plant with unknown sequence information thereof can be obtained by cloning with the use of the information of a plant gene with a known sequence. Methods for obtaining a desired gene via cloning is known in the molecular biological field. For example, when a gene sequence is known, an appropriate genomic library is constructed through digestion with restriction endonuclease, and then screening can be performed using a probe complementary to the desired gene sequence. When a sequence is isolated, DNA is amplified by a standard amplification method such as polymerase chain reaction (PCR), and thus the gene (DNA) can be obtained in an amount appropriate for transformation (gene transfer). A vector to be used herein may be a vector generally known for plant transformation. For example, a binary vector or other vectors can be used. A binary vector contains two border sequences (about 25-bp right border (RB) and about 25-bp left border (LB)) of Agrobacterium T-DNA. Between both border sequences, the gene encoding the above polypeptide is inserted. Examples of such a binary vector include pBI vectors (for example, pBI101, pBI101.2, pBI101.3, pBI121 and pBI221 (these vectors are manufactured by Clontech)), pGA482, pGAH and pBIG. Examples of other vectors include intermediate plasmids pLGV23Neo, pNCAT and pMON200, and pH35GS containing a GATEWAY cassette (Kubo et al., 2005, Genes & Dev. 19: 1855-1860). Examples of a promoter that can be used herein include, but are not particularly limited to, as long as it can activate gene expression in plant cells, a cauliflower mosaic virus 35S promoter (CaMV 35S), various actin gene promoters, various ubiquitin gene promoters, a nopaline synthase gene promoter, a tobacco PR1a gene promoter, a napin gene promoter, and an oleosin gene promoter. A selection marker required for selection of transformed cells is further inserted into a vector. Examples of a selection marker include drug resistance genes such as a kanamycin resistance gene, a hygromycin resistance gene, and a bialaphos resistance gene.

An example of a transformation method involving introducing the thus constructed vector into a plant is a method using Agrobacterium. Other than this method, a vector can be introduced using a gene gun, electroporation, viral vectors, a floral dip method, a leaf disk method, or the like. Plant transformation techniques and tissue culture techniques are described in Plant Cell Engineering Series 15, Experimental Protocols for Model Plants, Genetic Techniques to Genome Analysis (supervised by Isao Shimamoto and Kiyotaka Okada, Shujunsha (2001)), for example.

A method utilizing a binary vector-agrobacterium system involves preparing plant cells, calluses, or plant tissue pieces, infecting these with Agrobacterium, and then introducing the gene encoding the polypeptide into plant cells. Transformation may involve adding a phenol compound (acetosyringone) to a medium. Particularly in the case of monocotyledons, the cells can be transformed efficiently. In addition, Agrobacterium tumefaciens strains (e.g., C58, LBA4404, EHA101, EHA105, C58C1RifR and GV3101) can be used as Agrobacterium.

Specifically, an Agrobacterium suspension is prepared by about 4 days of culture in the dark at about 25° C. Plant calluses or tissues (e.g., laminae, roots, stem sections, or growing points) are immersed in the bacterial suspension for several minutes, water is removed, and then the resultants are placed on a solid medium for cocultivation. A callus is a mass of plant cells, which can be induced using a callus induction medium from a plant tissue section, a fully ripened seed or the like. The transformed calluses or tissue sections are selected based on a selection marker. Calluses can subsequently be caused to redifferentiate into adventitious shoots using a redifferentiation medium. Meanwhile, regarding plant sections, calluses can be induced from the plant sections and then induced and caused to redifferentiate into adventitious shoots, or protoplasts can be prepared from the plant sections and then caused to redifferentiate into adventitious shoots after callus culture. The thus obtained adventitious shoots are transplanted into soil after rooting so that they are regenerated into plants.

Further, when a floral dip method is used, the method is performed, for example, as described by Clough and Bent et al. (Plant J. 16, 735-743 (1998)), for example, as follows: an Agrobacterium suspension is prepared by about 4 days of culture in the dark at about 25° C., floral buds of the plant host to be transformed, which have been grown until the development of immature floral buds, are immersed in the bacterial suspension for 10 seconds, and then left to stand overnight with a cover to keep humidity; the cover is removed on the next day, the plants are allowed to grow and then seeds are harvested; transformed individual plants can be selected by seeding harvested seeds on a solid medium supplemented with an appropriate selection marker such as an antibiotic; the thus selected individual plants are transplanted into soil and allowed to grow, so that next-generation seeds of the transgenic plants can be obtained.

Progeny plants having a novel phenotype similar to that of transgenic plants can be produced by crossing the transgenic plants with wild-type plants.

As a host plant, any plant that is affected by the above polypeptide(s) can be used. Examples of such a plant include, but are not limited to, the above defined plants.

Transgenic plants are capable of expressing one or a plurality of the above polypeptides. The phenotype is changed and/or environmental stress resistance is enhanced depending on the activity of one or a plurality of overexpressed polypeptides.

The present invention is mainly described using transgenic plants overexpressing polypeptides. Useful traits can also be obtained by suppressing the expression of such polypeptides. The expression can be suppressed according to a conventionally known method such as gene disruption, a method using RNAi, a method using antisense RNA, and a method using ribozymes.

In addition, examples of the term “transgenic plant(s)” as used herein include the entire plant bodies of transgenic plants (e.g., sprouts and seedlings), plant portions (e.g., organs and tissues), seeds, calluses, cells, and/or shoots.

The present invention also relates to an agricultural composition containing a polypeptide having the activity to control plant morphology or a polypeptide having the activity to enhance the environmental stress resistance of plants.

The agricultural composition of the present invention contains one or a plurality of the above polypeptides as active ingredients.

The term “agricultural” as used herein refers to the cultivation and production of cereals, vegetables, fruit trees and horticultural crops. The agricultural composition of the present invention can be used for various useful plants. Examples of such useful plants include, but are not limited to, the above defined plants.

The above polypeptides can be obtained from the transgenic plants overexpressing the polypeptides. Specifically, after the pulverization of transgenic plants overexpressing the target polypeptides by known methods such as ultrasonic treatment, pulverization using a French press, a millstone, or a mortar, crushing using a homogenizer, pulverization using glass beads, the polypeptides can be extracted and/or purified using one of or a combination of a plurality of known methods that are generally employed for protein extraction, such as salting out with ammonium sulfate, precipitation separation using organic solvents (ethanol, methanol, acetone, etc.), chromatography (e.g., ion exchange chromatography, isoelectric chromatography, gel filtration chromatography, hydrophobic chromatography, adsorption column chromatography, affinity chromatography using substrates, antibodies, or the like, and reverse phase column chromatography), and filtration (e.g., microfiltration, ultrafiltration, and reverse osmosis filtration).

Alternatively, polypeptides secreted into a medium can be collected by culturing transgenic plants overexpressing the target polypeptides in the medium. A medium that can be used herein may be a liquid or solid medium, and is preferably a liquid medium. As described in the following examples, transgenic plants are cultured in an appropriate plant culture medium (e.g., MS medium), the medium is collected, and then the target polypeptide(s) can be extracted and/or purified from the collected medium. Culture can be performed by batch culture or continuous culture.

Alternatively, the above polypeptides can also be produced using a chemical technique (e.g., a solid phase synthesis method and a liquid phase synthesis method), or a transgenic technique using microbial cells (for example, E. coli and yeast), insect cells or animal cells (e.g., SF9, SF21, COS1, COST, CHO, and HEK293) transformed to express the target polypeptides.

The resulting polypeptides may be prepared to be dissolved or suspended in a suitable solvent, or prepared into a dry powdery form by drying the polypeptides using a general drying method (for example, natural drying, heat drying, and lyophilization).

The composition of the present invention can take a variety of forms such as a liquid preparation and a solid preparation (e.g., tablets, powders and granules) and is preferably in the form of a liquid preparation.

The composition can contain, in addition to the above polypeptides, and one or more types of other agriculturally acceptable ingredients. Examples of such ingredients include, but are not limited to, water, additional nutrient substances, weak acid, vegetable oils, essential oils, metabolic stimulating agents, emulsifiers, thickeners, coloring agents, suspending agents, dispersing agents, carriers, excipients, and wetting agents. The composition of the present invention can further contain fertilizers, pesticides, fungicides, and nematicides, as needed.

The agricultural compositions of the present invention can contain each of the above polypeptides in an amount adequately selected from the range of 0.01 to 95% by weight, for example.

The present invention further relates to a method for growing plants, comprising applying a polypeptide having the activity to control plant morphology or a polypeptide having the activity to enhance the stress resistance of plants to plants.

One of or a plurality of the above polypeptides, or the above agricultural compositions are applied to plants, so that the plant morphology is controlled, and/or the environmental stress resistance can be enhanced or imparted to the plants, depending on the activity of one of or a plurality of the polypeptides that are active ingredients.

The method of the present invention is used for various useful plants. Examples of target useful plants include, but are not limited to, the above defined plants.

The method to be applied herein can be adequately selected depending on the forms of the above polypeptides or the above agricultural compositions or plants. For example, plants, soil, or media can be directly subjected to high-pressure or low-pressure spraying, injection, spread, coating, infiltration, or the like. The soil and the medium can contain a moisturizer (any moisturizer can be used herein, as long as it can retain water, such as filter paper, fiber, wood, and gel made of polyvinyl alcohol (PVA), cellophane, cellulose acetate, cellulose nitrate, ethyl cellulose, or polyesters (examples thereof are not limited thereto)). Such a moisturizer may retain the above polypeptides or the above agricultural compositions. The method can be applied to various forms of plants, including entire plant bodies (e.g., sprouts and seedlings) and plant portions (for example, organs and tissues), seeds, calluses, cells, and shoots. The concentration for application; that is, the concentration of the above polypeptide that is an active ingredient, which can be employed herein, is appropriately selected from the range of 0.001 ppm-1000 ppm, 0.01 ppm-100 ppm, or 0.1 ppm-100 ppm. The concentrations (amounts) are not limited to these ranges and can be adequately regulated.

The present invention will be further described specifically by referring to the following examples. However, the present invention is not limited by these examples.

### Example 1

### Identification of Candidate Genes

Candidate genes were identified according to the method described in Hanada et al, BIOINFORMATICS, Vol. 26, no 3, 2010, pages 399-400. After identification of 7901 short genes encoding 30 to 100 amino acid residues on the basis of Arabidopsis (Arabidopsis thaliana)-derived 96358 full-length cDNAs (RIKEN Arabidopsis full-length (RAFL) cDNA resources (http://www.brc.riken.jp/lab/epd/catalog/cDNAclone.html) and NCBI Genbank (http://www.ncbi.nlm.nih.gov/genbank/)), the expression of these genes in Arabidopsis thaliana was analyzed using microarrays.

As a result, the expression of 4664 genes was confirmed. Eight hundred and ninety-one (891) genes randomly selected therefrom were used for preparation of the following overexpression plants.

### Example 2

### Preparation of Gene Overexpression Plants and Screening Based on Phenotypes

After amplification of the 891 genes obtained in Example 1 by PCR, the resultants were each ligated under the control of a double 35S promoter. The resultants were each introduced into a pMDC32 vector, and thus a recombinant binary vector was constructed.

Subsequently, the vector was introduced into Agrobacterium tumefaciens (GV3101 strain) and then Arabidopsis was infected with the thus obtained Agrobacterium using a floral dip method. Transformed seedlings were selected on a 50% MS medium (containing 20 mg 1⁻¹ hygromycin B, 100 mg 1⁻¹ cefotaxime), and then transplanted into soil. The prominent phenotype of each seedling was monitored.

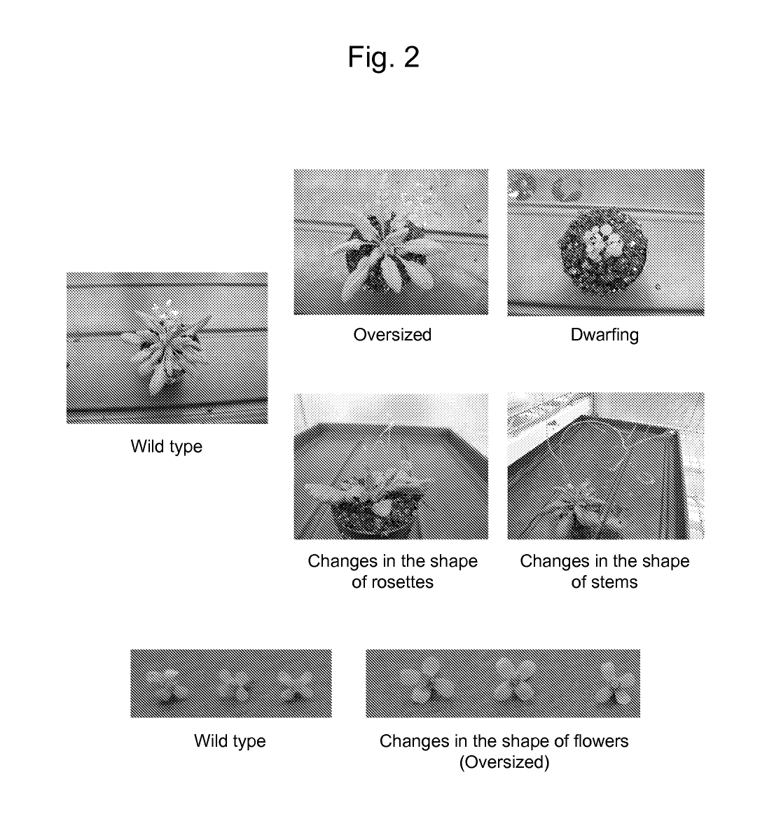

The phenotype of each seedling and the gene introduced therein are shown in Table 1 below. Typical changes in phenotype are shown in FIG. 2.

TABLE 1

| Phenotype | | Transgene (ID NO) | | | | | Total |
|---|---|---|---|---|---|---|---|
| Growth | Oversized | sORF44 | sORF273 | sORF367 | sORF618 | sORF711 | 21 |
| | | sORF714 | sORF726 | sORF774 | sORF791 | sORF801 | |
| | | sORF813 | sORF890 | AG232 | AG234 | AG236 | |
| | | AG237 | AG239 | flc8 | flc17 | flc21 | |
| | | flc39 | | | | | |
| | Dwarfing | sORF84 | sORF86 | sORF201 | sORF248 | sORF866 | 14 |
| | | sORF109 | sORF165 | sORF278 | sORF314 | AG4 | |
| | | AG53 | AG87 | AG96 | AT32 | | |
| Flowering | Delayed | sORF361 | sORF677 | sORF755 | sORF940 | flc30 | 6 |
| time | | AG156 | | | | | |
| Rosette | Changes | sORF108 | sORF126 | sORF169 | AG77 | AG110 | 8 |
| | in shape | AT5 | AT23 | AT33 | | | |
| | Darkening | sORF145 | sORF330 | sORF655 | sORF891 | | 4 |
| | Lightening | sORF59 | sORF70 | sORF389 | | | 3 |
| | Changes | | | | | | |
| | in number | | | | | | |
| Cauline | Changes | | | | | | |
| | in shape | | | | | | |
| | Changes | | | | | | |
| | in color tone | | | | | | |
| | Changes | | | | | | |
| | in number | | | | | | |
| Stem | Changes | sORF541 | sORF648 | | | | 2 |
| | in shape | | | | | | |
| | Changes | | | | | | |
| | in color tone | | | | | | |
| Flower | Changes | sORF695 | | | | | 1 |
| | in shape | | | | | | |
| | Other | | | | | | |
| Silique | Changes | sORF880 | | | | | 1 |
| | in shape | | | | | | |
| | Acarpous | sORF826 | sORF895 | | | | 2 |
| | Death | sORF47 | sORF147 | sORF284 | sORF576 | sORF740 | 7 |
| | | sORF759 | sORF783 | | | | |

| | |
|---|---|
| Total | 69 |

As shown in Table 1, a change in phenotype was observed in the 69 overexpression plants overexpressing each gene.

### Example 3

### Screening by Stress Resistance Test

Forty (40) overexpression plants from among the 891 overexpression plants (prepared in Example 2) were subjected to the following stress resistance test.

Screening by a stress resistance test was performed by conducting stress (3 stresses: drought, salt, and high temperature) resistance tests. Each test was conducted by setting conditions where wild-type plants exhibit the survival rate of about 10%-30%, comparing the wild-type plants with overexpression plants, and then evaluating resistance based on the resulting survival rates. Each test method is as specifically described below.

Drought Stress Resistance Test

The drought resistance test was conducted by growing the plants for 10 days after sowing onto an MS plate, transplanting the plants in soil (pot), and then growing the plants for 7 days for the plants to take roots. Water supply was stopped to gradually create a drought condition. After 14 days of stopping water supply, water supply was restarted. The plants were grown for 7 days and then the survival rates were observed.

Salt Stress Resistance Test

The salt stress resistance test was conducted by performing salt treatment that involves transferring plants that had been grown for 7 days after sowing on an MS plate to an MS plate containing 200 mM NaCl. The plants were grown for 14 days, and then the survival rates and the effects of the salt stress were observed.

High-Temperature Stress Resistance Test

The high-temperature resistance test was conducted by performing high-temperature treatment for 3 hours, which involves transferring plants that had been grown for 14 days after sowing on an MS plate to an air tank incubator at 42° C., growing the plants for 7 days at 22° C., and then observing the survival rates. Specifically the high-temperature resistance test was conducted by sowing wild-type plants and overexpression plants on the same plate.

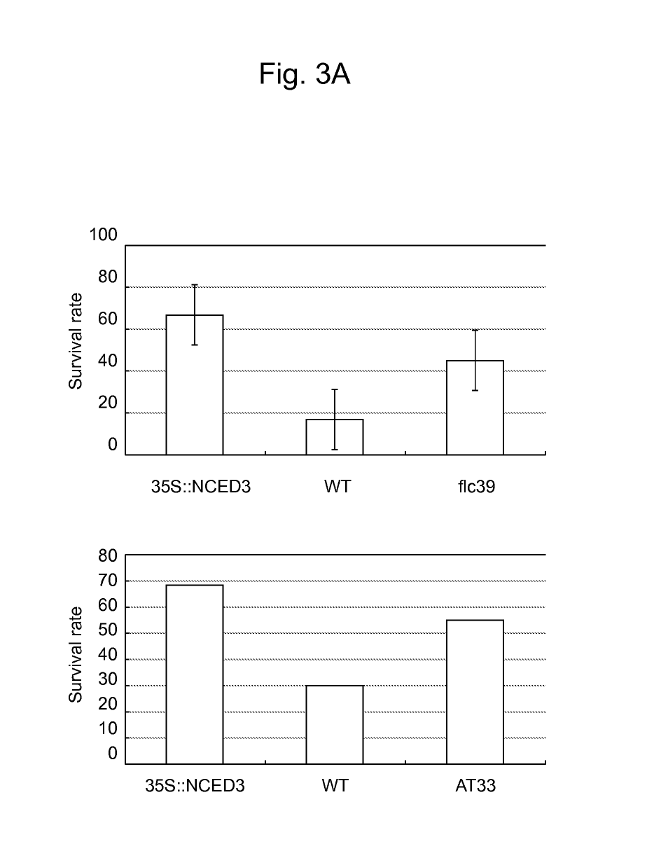

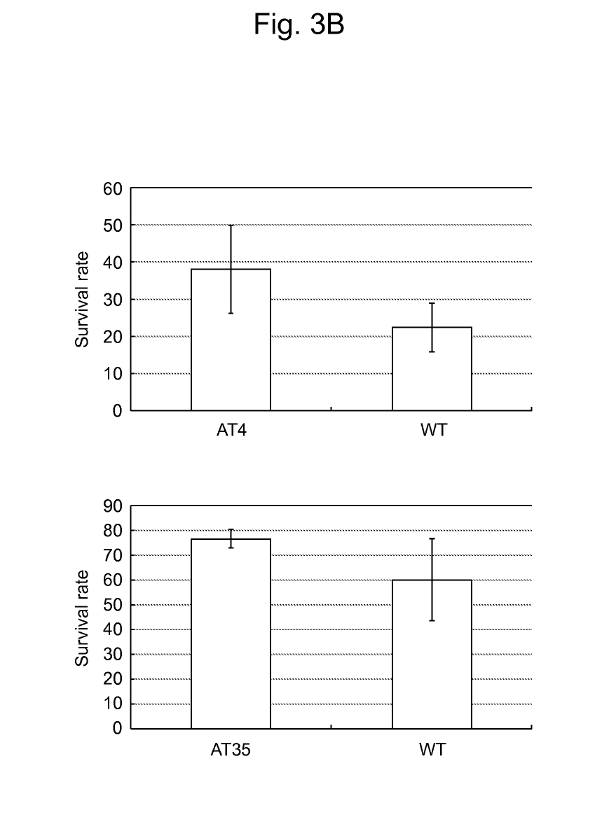

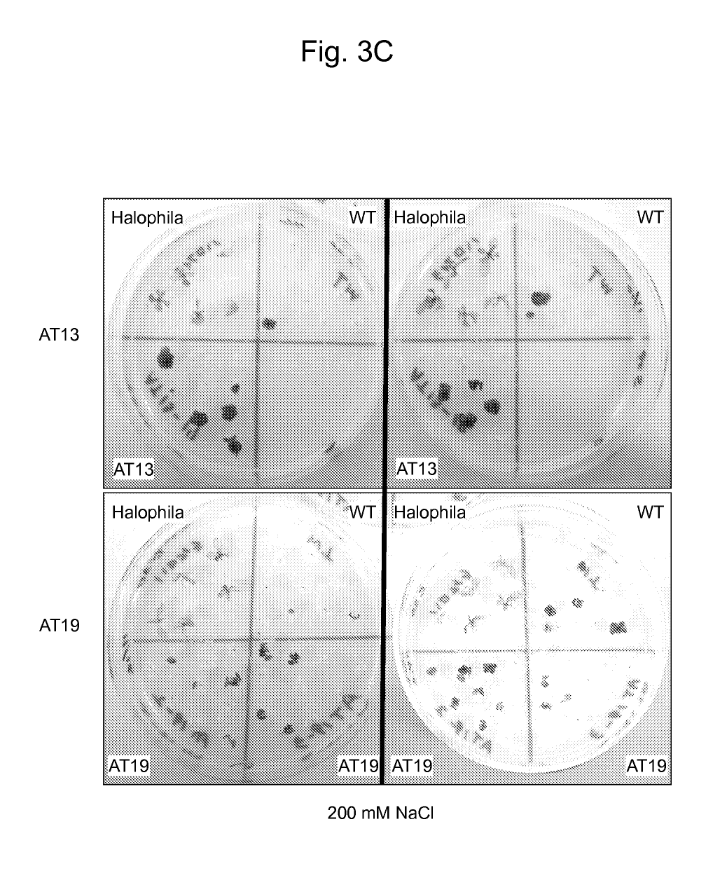

The phenotype of the stress resistance of each seedling and the gene introduced therein are shown in Table 2 below. In addition, the relationship between each stress and the fatality rate of each overexpression plant is shown in FIG. 3A-C.

TABLE 2

| Phenotype | Transgene (ID NO) | | | Total |
|---|---|---|---|---|
| Drought resistance | flc39 | AT33 | | 2 |
| High temperature resistance | AT4 | AT35 | AT36 | 3 |
| Salt resistance | AT13 | AT19 | AT56 | 3 |

| | |
|---|---|
| Total | 8 |

As shown in Table 2, stress resistance was observed in the 8 overexpression plants overexpressing each gene.

### Example 4

### GUS Analysis

The expression of the 69 genes that had been introduced into seedlings (for which changes in phenotype were observed in Example 2) and the 8 genes that had been introduced into seedlings (for which changes in stress resistance were observed in Example 3) was analyzed using a GUS fusion.

Genomic fragments, each containing ORF excluding an about 1-Kb upstream portion of the start codon and the termination codon of each gene, were amplified by PCR. A sequence required for GATEWAY (Invitrogen) cloning was added to an end of each of the obtained PCR products. The resultant was cloned to a GATEWAY vector (pDONR207) and then inserted to a GATEWAY vector (pMDC162), so that ORF and a GUS gene were fused. The thus prepared constructs were transformed into Arabidopsis by an Agrobacterium method. Analysis was conducted by a GUS staining method using each organ of the thus obtained transformants.

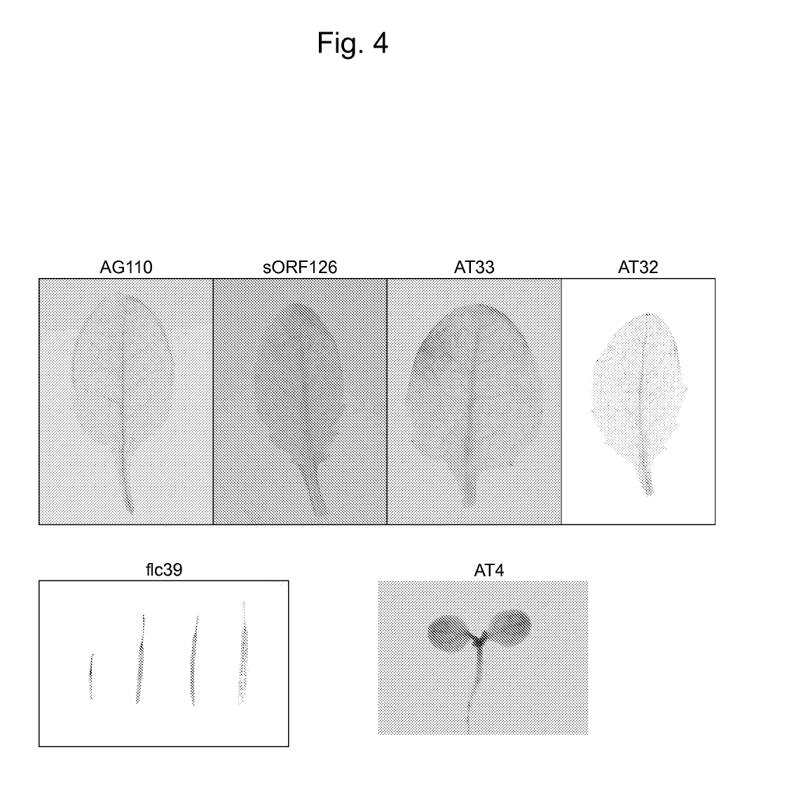

Typical results of GUS analysis of each gene are shown in FIG. 4.

As shown in FIG. 4, GUS was stained and detected in each organ (e.g., leaves, sheaths, and sprouts) of each transformant, and thus the expression of these genes in plant bodies was confirmed.

### Example 5

### Gene Function Suppression Test Using RNAi

Sixty nine (69) genes that had been introduced into seedlings (for which changes in phenotype were observed in Example 2) and the 8 genes that had been introduced into seedlings (for which changes in stress resistance were observed in Example 3) were subjected to a gene function suppression test using RNAi molecules targeting the genes.

RNAi molecules were prepared based on a conventionally known technique. Specifically, each oligonucleotide was synthesized, wherein the sense and antisense strands of each gene were ligated via a loop sequence (linker sequence) in directions opposite to each other.

Each RNAi molecule for each gene was amplified by PCR, and then a sequence required for GATEWAY cloning was added to an end of each of the thus obtained PCR products. The PCR product was cloned to a GATEWAY vector (pDONR207). Moreover, the ORF was inserted to a GATEWAY overexpression vector (pMDC32), transformation was performed in a manner similar to that in Example 2 above, and thus transgenic plants, in which the target genes were suppressed by the RNAi molecules, were prepared.

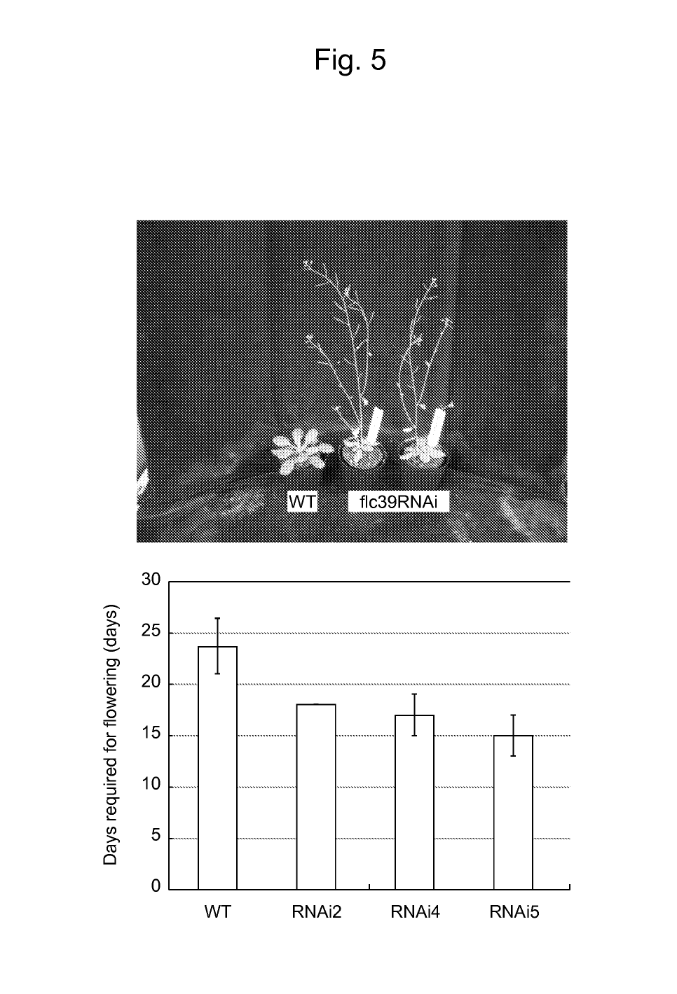

The typical results of the gene function suppression test for each gene using RNAi are shown in FIG. 5. As shown in FIG. 5, changes in the growth of plant bodies and the flowering times were observed by suppressing the function of the flc39 gene.

### Example 6

### Addition Test Using Liquid Medium of Overexpression Plants

The sprouts of the 69 overexpression plants (for which changes in phenotype were observed in Example 2) and the 8 overexpression plants (for which changes in stress resistance were observed in Example 3) were each grown on MS agar media containing hygromycin for 2 weeks. Subsequently, individual plants that had become resistant to hygromycin, and specifically about 30 plants overexpressing each gene, were transferred into a 300-ml flask containing 100 ml of an MS+1% sucrose liquid medium. The plants were then grown for 1 week. Subsequently, the MS+1% sucrose liquid medium was exchanged with 100 ml of a sucrose-free 1/10 MS liquid medium. The plants were grown in this condition for 1 week.

Next, the medium was collected, lyophilized, and then dissolved in 10 ml of sterile water. In addition, the concentration of MS in the solution is normal concentration (the amount of the secreted peptide should theoretically be 10 times greater than that before lyophilization). One (1) mL of the solution was added to filter paper placed on a sucrose-free MS agar medium, and then wild-type seeds were sown, 25 seeds per Petri dish. The seeds were left to stand in the dark at 4° C. and then germinated at 22° C. under the long-day conditions. Two days after germination, the solution was added again, the plants were grown for 2 weeks, and then the morphology was observed.

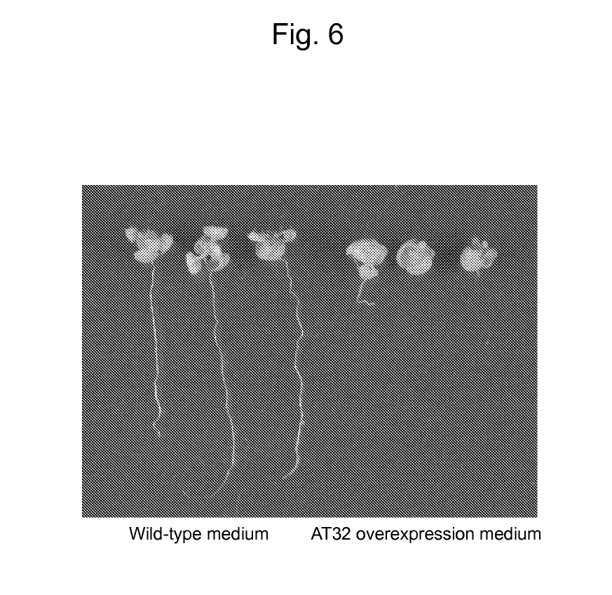

Typical results of the addition test for each gene are shown in FIG. 6.

As shown in FIG. 6, as a result of the addition test using the liquid medium of AT32-overexpressing plants, dwarfing was observed in wild-type sprouts. The AT32-overexpressing plants themselves exhibited the dwarfing phenotype (Table 1), demonstrating that AT32 was secreted into the medium while retaining its functions.

Next, rice seeds were similarly treated using the liquid medium of overexpression plants similarly obtained as described above.

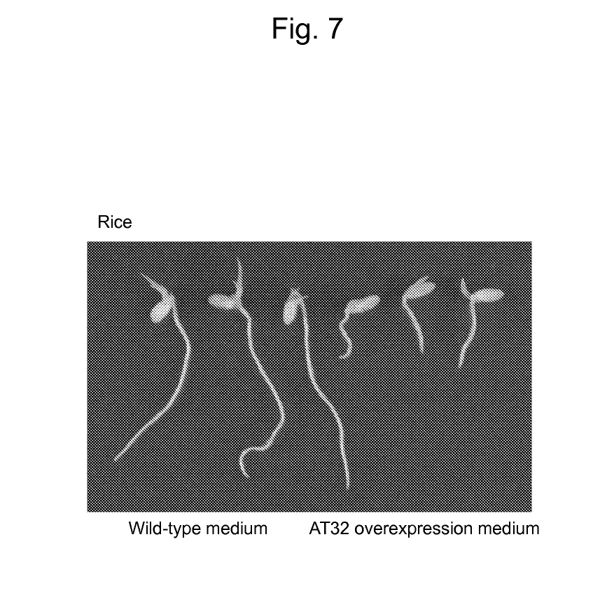

Typical results of the addition test for each gene are shown in FIG. 7.

As shown in FIG. 7, as a result of the addition test using the liquid medium of AT32-overexpressing plants, dwarfing was also observed in rice sprouts. This result demonstrates that peptides obtained from Arabidopsis can be similarly activated and function in other plants such as rice.

### INDUSTRIAL APPLICABILITY

According to the present invention, plants having a desired phenotype and/or desired stress resistance can be obtained. Furthermore, according to the present invention, a desired phenotype and/or desired stress resistance can be imparted to plants. Therefore, useful crops can be efficiently produced and the present invention is expected to contribute to various fields relating to the production and/or the use of useful crops.

All publications, patents, and patent applications cited herein are incorporated herein by reference in their entirety.

1. A method for producing a transgenic plant, comprising introducing a gene encoding a polypeptide that has the activity to control plant morphology or a polypeptide that has the activity to enhance the environmental stress resistance of plants into a host plant and then causing the overexpression thereof, wherein the polypeptide(s) is one polypeptide or are a plurality of polypeptides selected from the following (a) to (d):
(a) a polypeptide comprising any one of the amino acid sequences represented by SEQ ID NOS: 21, 1-20 and 22-317;
(b) a polypeptide comprising an amino acid sequence that has a deletion, a substitution, an addition or an insertion of 1 or several amino acids with respect to any one of the amino acid sequences represented by SEQ ID NOS: 21, 1-20 and 22-317, and having the activity to control plant morphology or the activity to enhance the environmental stress resistance of plants;
(c) a polypeptide comprising an amino acid sequence that has 60% or more identity with any one of the amino acid sequences represented by SEQ ID NOS: 21, 1-20 and 22-317, and having the activity to control plant morphology or the activity to enhance the environmental stress resistance of plants; and
(d) a polypeptide, which is obtained by the expression of a gene encoding any one of the polypeptides (a) to (c) in a plant cell and has the activity to control plant morphology or the activity to enhance the environmental stress resistance of plants.

2. The method according to claim 1, wherein the polypeptide(s) has the activity to control plant morphology and is one polypeptide or are a plurality of polypeptides selected from the following (e) to (h):
(e) a polypeptide comprising any one of the amino acid sequences represented by SEQ ID NOS: 21, 1-20 and 22-69;
(f) a polypeptide comprising an amino acid sequence that has a deletion, a substitution, an addition, or an insertion of 1 or several amino acids with respect to any one of the amino acid sequences represented by SEQ ID NOS: 21, 1-20 and 22-69, and having the activity to control plant morphology;
(g) a polypeptide comprising an amino acid sequence that has 60% or more identity with any one of the amino acid sequences represented by SEQ ID NOS: 21, 1-20 and 22-69, and having the activity to control plant morphology; and
(h) a polypeptide, which is obtained by the expression of a gene encoding any one of the polypeptides (e) to (g) in a plant cell, and has the activity to control plant morphology.

3. The method according to claim 1, wherein the polypeptide(s) has the activity to enhance the environmental stress resistance of plants and is one polypeptide or are a plurality of polypeptides selected from the following (i) to (l):
(i) a polypeptide comprising any one of the amino acid sequences represented by SEQ ID NOS: 21, 15 and 70-75;
(j) a polypeptide comprising an amino acid sequence that has a deletion, a substitution, an addition or an insertion of 1 or several amino acids with respect to any one of the amino acid sequences represented by SEQ ID NOS: 21, 15 and 70-75, and having the activity to enhance the environmental stress resistance of plants;
(k) a polypeptide comprising an amino acid sequence that has 60% or more identity with any one of the amino acid sequences represented by SEQ ID NOS: 21, 15 and 70-75, and having the activity to enhance the environmental stress resistance of plants;
(l) a polypeptide, which is obtained by the expression of a gene encoding any one of the polypeptides (i) to (k) in a plant cell and has the activity to enhance the environmental stress resistance of plants.

4. A transgenic plant overexpressing a polypeptide that has the activity to control plant morphology or a polypeptide that has the activity to enhance the environmental stress resistance of plants, wherein the polypeptide(s) is one polypeptide or are a plurality of polypeptides selected from the following (a) to (d):
(a) a polypeptide comprising any one of the amino acid sequences represented by SEQ ID NOS: 21, 1-20 and 22-317;
(b) a polypeptide comprising an amino acid sequence that has a deletion, a substitution, an addition or an insertion of 1 or several amino acids with respect to any one of the amino acid sequences represented by SEQ ID NOS: 21, 1-20 and 22-317, and having the activity to control plant morphology or the activity to enhance the environmental stress resistance of plants;
(c) a polypeptide comprising an amino acid sequence that has 60% or more identity with any one of the amino acid sequences represented by SEQ ID NOS: 21, 1-20 and 22-317, and having the activity to control plant morphology or the activity to enhance the environmental stress resistance of plants;
(d) a polypeptide, which is obtained by the expression of a gene encoding any one of the polypeptides (a) to (c) in a plant cell and has the activity to control plant morphology or the activity to enhance the environmental stress resistance of plants.

5. The transgenic plant according to claim 4, wherein the polypeptide(s) has the activity to control plant morphology and is one polypeptide or are a plurality of polypeptides selected from the following (e) to (h):
(e) a polypeptide comprising any one of the amino acid sequences represented by SEQ ID NOS: 21, 1-20 and 22-69;
(f) a polypeptide comprising an amino acid sequence that has a deletion, a substitution, an addition or an insertion of 1 or several amino acids with respect to any one of the amino acid sequences represented by SEQ ID NOS: 21, 1-20 and 22-69, and having the activity to control plant morphology;
(g) a polypeptide comprising an amino acid sequence that has 60% or more identity with any one of the amino acid sequences represented by SEQ ID NOS: 21, 1-20 and 22-69, and having the activity to control plant morphology;
(h) a polypeptide, which is obtained by the expression of a gene encoding any one of the polypeptides (e) to (g) in a plant cell and has the activity to control plant morphology.

6. The transgenic plant according to claim 4, wherein the polypeptide(s) has the activity to enhance the environmental stress resistance of plants and is one polypeptide or are a plurality of polypeptides selected from the following (i) to (l):
(i) a polypeptide comprising any one of the amino acid sequences represented by SEQ ID NOS: 21, 15 and 70-75;
(j) a polypeptide comprising an amino acid sequence that has a deletion, a substitution, an addition or an insertion of 1 or several amino acids with respect to any one of the amino acid sequences represented by SEQ ID NOS: 21, 15 and 70-75, and having the activity to enhance the environmental stress resistance of plants;
(k) a polypeptide comprising an amino acid sequence that has 60% or more identity with any one of the amino acid sequences represented by SEQ ID NOS: 21, 15 and 70-75, and having the activity to enhance the environmental stress resistance of plants;
(l) a polypeptide, which is obtained by the expression of a gene encoding any one of the polypeptides (i) to (k) in a plant cell and has the activity to enhance the environmental stress resistance of plants.

7. An agricultural composition, containing a polypeptide that has the activity to control plant morphology or a polypeptide that has the activity to enhance the environmental stress resistance of plants, wherein the polypeptide(s) is one polypeptide or are a plurality of polypeptides selected from the following (a) to (d):
(a) a polypeptide comprising any one of the amino acid sequences represented by SEQ ID NOS: 21, 1-20 and 22-317;
(b) a polypeptide comprising an amino acid sequence that has a deletion, a substitution, an addition or an insertion of 1 or several amino acids with respect to any one of the amino acid sequences represented by SEQ ID NOS: 21, 1-20 and 22-317, and having the activity to control plant morphology or the activity to enhance the environmental stress resistance of plants;
(c) a polypeptide comprising an amino acid sequence that has 60% or more identity with any one of the amino acid sequences represented by SEQ ID NOS: 21, 1-20 and 22-317, and having the activity to control plant morphology or the activity to enhance the environmental stress resistance of plants;
(d) a polypeptide, which is obtained by the expression of a gene encoding any one of the polypeptides (a) to (c) in a plant cell and has the activity to control plant morphology or the activity to enhance the environmental stress resistance of plants.

8. The composition according to claim 7, wherein the polypeptide(s) has the activity to control plant morphology and is one polypeptide or are a plurality of polypeptides selected from the following (e) to (h):
(e) a polypeptide comprising any one of the amino acid sequences represented by SEQ ID NOS: 21, 1-20 and 22-69;
(f) a polypeptide comprising an amino acid sequence that has a deletion, a substitution, an addition or an insertion of 1 or several amino acids with respect to any one of the amino acid sequences represented by SEQ ID NOS: 21, 1-20 and 22-69, and having the activity to control plant morphology;
(g) a polypeptide comprising an amino acid sequence that has 60% or more identity with any one of the amino acid sequences represented by SEQ ID NOS: 21, 1-20 and 22-69, and having the activity to control plant morphology;
(h) a polypeptide, which is obtained by the expression of a gene encoding any one of the polypeptides (e) to (g) in a plant cell and has the activity to control plant morphology.

9. The composition according to claim 7, wherein the polypeptide(s) has the activity to enhance the stress resistance of plants and is one polypeptide or are a plurality of polypeptides selected from the following (i) to (l):
(i) a polypeptide comprising any one of the amino acid sequences represented by SEQ ID NOS: 21, 15 and 70-75;
(j) a polypeptide comprising an amino acid sequence that has a deletion, a substitution, an addition or an insertion of 1 or several amino acids with respect to any one of the amino acid sequences represented by SEQ ID NOS: 21, 15 and 70-75, and having the activity to enhance the environmental stress resistance of plants;
(k) a polypeptide comprising an amino acid sequence that has 60% or more identity with any one of the amino acid sequences represented by SEQ ID NOS: 21, 15 and 70-75, and having the activity to enhance the environmental stress resistance of plants;
(l) a polypeptide, which is obtained by the expression of a gene encoding any one of the polypeptides (i) to (k) in a plant cell and has the activity to enhance the environmental stress resistance of plants.

10. A method for growing plants, comprising applying a polypeptide that has the activity to control plant morphology or a polypeptide that has the activity to enhance the stress resistance of plants to a plant, wherein the polypeptide(s) is one polypeptide or are a plurality of polypeptides selected from the following (a) to (d):
(a) a polypeptide comprising any one of the amino acid sequences represented by SEQ ID NOS: 21, 1-20 and 22-317;
(b) a polypeptide comprising an amino acid sequence that has a deletion, a substitution, an addition or an insertion of 1 or several amino acids with respect to any one of the amino acid sequences represented by SEQ ID NOS: 21, 1-20 and 22-317, and having the activity to control plant morphology or the activity to enhance the environmental stress resistance of plants;
(c) a polypeptide comprising an amino acid sequence that has 60% or more identity with any one of the amino acid sequences represented by SEQ ID NOS: 21, 1-20 and 22-317, and having the activity to control plant morphology or the activity to enhance the environmental stress resistance of plants;
(d) a polypeptide, which is obtained by the expression of a gene encoding any one of the polypeptides (a) to (c) in a plant cell and has the activity to control plant morphology or the activity to enhance the environmental stress resistance of plants.

11. The method according to claim 10, wherein the polypeptide(s) has the activity to control plant morphology and is one polypeptide or are a plurality of polypeptides selected from the following (e) to (h):
(e) a polypeptide comprising any one of the amino acid sequences represented by SEQ ID NOS: 21, 1-20 and 22-69;
(f) a polypeptide comprising an amino acid sequence that has a deletion, a substitution, an addition or an insertion of 1 or several amino acids with respect to any one of the amino acid sequences represented by SEQ ID NOS: 21, 1-20 and 22-69, and having the activity to control plant morphology;
(g) a polypeptide comprising an amino acid sequence that has 60% or more identity with any one of the amino acid sequences represented by SEQ ID NOS: 21, 1-20 and 22-69, and having the activity to control plant morphology;
(h) a polypeptide, which is obtained by the expression of a gene encoding any one of the polypeptides (e) to (g) in a plant cell and has the activity to control plant morphology.

12. The method according to claim 10, wherein the polypeptide(s) has the activity to enhance the stress resistance of plants and is one polypeptide or are a plurality of polypeptides selected from the following (i) to (l):
(i) a polypeptide comprising any one of the amino acid sequences represented by SEQ ID NOS: 21, 15 and 70-75;
(j) a polypeptide comprising an amino acid sequence that has a deletion, a substitution, an addition or an insertion of 1 or several amino acids with respect to any one of the amino acid sequences represented by SEQ ID NOS: 21, 15 and 70-75, and having the activity to enhance the environmental stress resistance of plants;
(k) a polypeptide comprising an amino acid sequence that has 60% or more identity with any one of the amino acid sequences represented by SEQ ID NOS: 21, 15 and 70-75, and having the activity to enhance the environmental stress resistance of plants;
(l) a polypeptide, which is obtained by the expression of a gene encoding any one of the polypeptides (i) to (k) in a plant cell and has the activity to enhance the environmental stress resistance of plants.


SEQUENCE LISTING (317 sequences)

SEQ ID NO: 1 - protein, 147 aa, Arabidopsis thaliana
MEAIRFSNLSGLLILLMSLPLLVTSKDETVSCTMCSSCDNPCNPVPSSYSPPPPPPSSSGGGGSYYYSPPPPSSSGGVKYPPPYGGDGYGGYYPPPYYGNYGTPPPPNPIVPYFPFYYHTPPQGYSGSARLQNSLLFALFAVLLCFV

SEQ ID NO: 2 - protein, 103 aa, Arabidopsis thaliana
MVVALVCAFVPVFSVEEAEAKSLRDTCLVKITPKCALNIITVVFGNGTLIEACCNDLVQEGKVCHDNLIKYIADRPSLIGRETQYLKKRDEVWSHCVSISKTA

SEQ ID NO: 3 - protein, 64 aa, Arabidopsis thaliana
MPLLRFFFSKSEELTSLDEYIDHMGESQTSIYYLLKSDYSYFLEKVPQTTKVLYLVDSTDKIHS

SEQ ID NO: 4 - protein, 44 aa, Arabidopsis thaliana
MQKNGPTMGSISVYILRKAMLGGMWSTCRNAWVVARLVIHCFPI

SEQ ID NO: 5 - protein, 32 aa, Arabidopsis thaliana
MHLIVKLKESQTVASSDCILDCLAGPNVVVNI

SEQ ID NO: 6 - protein, 46 aa, Arabidopsis thaliana
MSIQQEMVEESSRKVLLFRYFCGGEMNGNDVDARPTKLNTFSLNAV

SEQ ID NO: 7 - protein, 38 aa, Arabidopsis thaliana
MKRKEKNHRIKDFLGWEREGMRRHVGENIRKLEKGETS

SEQ ID NO: 8 - protein, 93 aa, Arabidopsis thaliana
MYPTRPHYWRRRLSINRPQAFLLLILCLFFIHHCDASRFSSSSVFYRNPNYDHSNNTVRRGHFLGFLPRHLPVPASAPSRKHNDIGIQALLSP

SEQ ID NO: 9 - protein, 39 aa, Arabidopsis thaliana
MGLARSRYFMTVLIKGLRNGNELCDQSQVGLLHRSRAYV

SEQ ID NO: 10 - protein, 130 aa, Arabidopsis thaliana
MKNLFIFVILLSVCIRNTFGISTLLVKNELNNKVLGVRCRSKDDNLGDHILRVGQMTKNNFDDNVWRRTLFWCNLWKGPDFKLHVAFDAYRSQWKADIGPRYLWIAREDGIYYTQHPETPPKKKYDWPKA

SEQ ID NO: 11 - protein, 134 aa, Arabidopsis thaliana
MSQALLNFQLSSSSFSIPKTLIHSHSLKTLKTPKLNSRQFFSQCTASSSDGPKTLRTCKNCKTQFDPLLNNPRACRFHTAHFGGETKRKFESVYTGGTMDTPNSGKVLQYWHCCGSEDPFDSGCTASPHTTYDD

SEQ ID NO: 12 - protein, 125 aa, Arabidopsis thaliana
MASNTSFLFVTVTLLLVLNVSSRALPPVADSTNIAARLTGGGLMQCWDALYELKSCTNEIVLFFLNGETKLGYGCCNAVDVITTDCWPAMLTSLGFTLEETNVLRGFCQSPNSGGSSPALSPVKL

SEQ ID NO: 13 - protein, 69 aa, Arabidopsis thaliana
MKRQVMIFVMLVAFFVVFLDVKQVEAMRPFPTAADEIRFVFQALQRGPVSGSGPNGCTNIPRGTPRCHG

SEQ ID NO: 14 - protein, 91 aa, Arabidopsis thaliana
MAVSKTTMLIVLVAIILSCVSISNARQMQRPQNVECVGGECQPKHPQQYFGSCFRDSDCRNSCFPYCRYQKCHHHECICSLCSSEVAPSPK

SEQ ID NO: 15 - protein, 268 aa, Arabidopsis thaliana
MSRRASKRVRFSPDPEANDELIFPTHSSSRHGRRRVVVGIFSFSFSDSPATTKRLLRSIGDRVGKTFRYISFGRMNTKTTPSSSSNVSSSLYLMKSKSLNGSESHRAEAIEDCIEFLNSCSSLSRSNSISTWSRMSRRASKRVRFSPDPEANDELIFPTHSSSRHGRRRVVVGIFSFSFSDSPATTKRLLRSIGDRVGKTFRYISFGRMNTKTTPSSSSNVSSSLYLMKSKSLNGSESHRAEAIEDCIEFLNSCSSLSRSNSISTWSR

SEQ ID NO: 16 - protein, 102 aa, Arabidopsis thaliana
MARIGALILVLFISISQLASFSTARKFPVGIPSVIDGVIFSGEISAVSKKVTVVGCEGEDDHLTAGYSSYITGKFGSLVLNALPKGSVPASGPSKRINDVKT

SEQ ID NO: 17 - protein, 37 aa, Arabidopsis thaliana
MAHLELSIPCDGSTKQPTRPTYLKFENRSRALRPQCL

SEQ ID NO: 18 - protein, 30 aa, Arabidopsis thaliana
MKFLVLGFERLALFLMGGGRCYLWVGLGHL

SEQ ID NO: 19 - protein, 86 aa, Arabidopsis thaliana
MYLLAAFAANSQAGETLSPGSGIDWFRADSGSCCLDYRRIRGAVLGKEMTQLQKKKDRCRLPTRARSSIGGQEPWSGGKTEVVRIK

SEQ ID NO: 20 - protein, 30 aa, Arabidopsis thaliana
MIFIVGLKLLVYVSGVGRGAFPSLVVWFRH

SEQ ID NO: 21 - protein, 61 aa, Arabidopsis thaliana
MCAYFSDVVITLILQAKLDCMIELDGGSLIEKSLTILLHQKETPWYLVVRYILAFICYFFP

SEQ ID NO: 22 - protein, 72 aa, Arabidopsis thaliana
MLLVLRKTHRRRDEIYVDRRAQIFNVEIQRQIGHKLLMGQPVNAHRSHNLMRMKCMFYGFYKRSRVMFSCVL

SEQ ID NO: 23 - protein, 46 aa, Arabidopsis thaliana
MNKLSSSINNGSECLERFMLFVVEWYERLKSRASDVLVSLVWCIES

SEQ ID NO: 24 - protein, 95 aa, Arabidopsis thaliana
MHNHKRSEIPLYNNAPIYTLWPFPTPTIRDSLFLSKIADFNSQYWCTIATDSPDRRGDDHSTISNARERLQTKLDSELNQGNLKTKPDSELNQGN

SEQ ID NO: 25 - protein, 67 aa, Arabidopsis thaliana
MECMEMQSGWDQGFYFDLKKIEGVCRYFQELCQRAFWSVRGAGATLPTETTLLIQFIGVHICSKIRD

SEQ ID NO: 26 - protein, 67 aa, Arabidopsis thaliana
MPLFFMVPALCFIDRLDLTCIVMAIWDIRMIGERDEKCCIWRLESYYNMMVSPDKSSFSGTLITLLE

SEQ ID NO: 27 - protein, 71 aa, Arabidopsis thaliana
MFEKGNLVLDYAAKNDSCYEYASKLMKPIGVWFLMMLVPCSFLCLKLVLQPMFRIFRVGLLFRLCPVAKET

SEQ ID NO: 28 - protein, 40 aa, Arabidopsis thaliana
MHAHNRNYPLDYIYFSLPPNGYSSSPIGKLNIIDIHPSVY

SEQ ID NO: 29 - protein, 85 aa, Arabidopsis thaliana
MFRLIGIIKHWMTNIMKGREETTRQGIGKDQRASPMKVLMGMLTPETKLMLRKSNPVTNTMSIEHPITLGIPTRRIKTINQFGSA

SEQ ID NO: 30 - protein, 38 aa, Arabidopsis thaliana
MTHVNMAARERTKLKETEKGRGPGNTRQPRSPHLKMYT

SEQ ID NO: 31 - protein, 52 aa, Arabidopsis thaliana
MGHRSGFGRAALSDQGPGHTGPGTIQVRRTPEVTAMSRNLTHRPKRRENTRT

SEQ ID NO: 32 - protein, 51 aa, Arabidopsis thaliana
MWSKFSVHFYLTLFPNNTALHTHAPSSKVAFEVNPCSKKGKSKQHSHTHKL

SEQ ID NO: 33 - protein, 78 aa, Arabidopsis thaliana
MFRIVIGLLIWAFSMESSSRRVGIKQKKNIRRENSSGHVPDLNTTLHSTLLSGKQILPYPSMKRRPHWIRVVLKEVCH

SEQ ID NO: 34 - protein, 48 aa, Arabidopsis thaliana
MVLIHLIEELCEKFFNHPQALSYHCWTVDFATWHEFVILSHLIILRRR

SEQ ID NO: 35 - protein, 72 aa, Arabidopsis thaliana
MVTERKQMFLVDGVTWWWWSRKKKEGDDTDDRGKTVFFPSTKYSFSKSNTEKPKFKDLAKKSPVLTINPLIS

SEQ ID NO: 36 - protein, 42 aa, Arabidopsis thaliana
MLCFSPVFWVLMLLSALYLGGGFEANNDIDKYIQNNAFDSFF

SEQ ID NO: 37 - protein, 64 aa, Arabidopsis thaliana
MRDFEIQQGAKCEGTRMKDRIGTNGYWGATEENNTLHHPLLFYTYNNAIENHFKPSRTLSLFPS

SEQ ID NO: 38 - protein, 42 aa, Arabidopsis thaliana
MEHERRRNIKDNKEGGGGALPVRRQGVPQTERRRRQIADIMI

SEQ ID NO: 39 - protein, 61 aa, Arabidopsis thaliana
MKTKFGDLAKCRQSKGQFMTAAETLHQNIEAVDIKWNPPQFHLLIERNRNIGLFHSDDDTH

SEQ ID NO: 40 - protein, 55 aa, Arabidopsis thaliana
MCRACCNKEQEVCNFSCSVCNQRSVCEDLDKMDSAPELLPYCLREWMVQDYISVV

SEQ ID NO: 41 - protein, 67 aa, Arabidopsis thaliana
MSEMSIRKVGATSKLGLLELKKKNEPKGEVQEAQSGFKFDTTMGQRPCKDTRIFWYKLAIHILLQYS

SEQ ID NO: 42 - protein, 61 aa, Arabidopsis thaliana
MSFRLHEDEQVSVRYYGSGDGCHSYIVDNATLVFFFSPTSWKLLLNSYRYGYLQRTCIFTI

SEQ ID NO: 43 - protein, 68 aa, Arabidopsis thaliana
MIKKMGLMGHYCFVLEFKLQWERDKRVDLGESGTMARGHVMPIHKEEKEDMFLSYVIKSSHGVLVCHY

SEQ ID NO: 44 - protein, 95 aa, Arabidopsis thaliana
MPWMEHTQVPFGWMDQPTMRDPTVLKKFSKSKMLLHVGKKEESRCEAKETTVIDRGPYQLAMFALQTRTNKCHVKSYTTAKLRSTGDICGPLGVR

SEQ ID NO: 45 - protein, 44 aa, Arabidopsis thaliana
MKPTVELTIDAQLLLHHLNQNFDSDLAWLIFFFDEASDYHPYLF

SEQ ID NO: 46 - protein, 48 aa, Arabidopsis thaliana
MGQPDPDLDPFNIARGQPVWNTPVQSRENHDMSQHSPSRPVSWQLSCL

SEQ ID NO: 47 - protein, 57 aa, Arabidopsis thaliana
MKLINVNLLFLPTSTYIDSGTRHDATWPYHHHSSPGSLVFVKPSIIIVSTTSYVSYI

SEQ ID NO: 48 - protein, 69 aa, Arabidopsis thaliana
MMHLAREIGDFNWMFVFMAGARITYGRSSVTRRMGEIGSFAGVDSEKLFLCINLRRRRPNLRYFFLYFS

SEQ ID NO: 49 - protein, 63 aa, Arabidopsis thaliana
MQQTITFGDKVALESSWGVAFDFACGGKFTDKKFRTCKVCEKTQIRYTELVNMLTCGSGPKHI

SEQ ID NO: 50 - protein, 55 aa, Arabidopsis thaliana
MKKEITIQLLAIPRAASISPVIPMDHQIMMTSNHLRSRVLAHVPVIRLEEFEIAI

SEQ ID NO: 51 - protein, 78 aa, Arabidopsis thaliana
MSHCPFLQFCSFCNGSVSSLFLFINSFVFCDKLLSGLDGPFGLNVVIGLNNWAQSQSFTKLSFELLTNILDIHPRFEF

SEQ ID NO: 52 - protein, 73 aa, Arabidopsis thaliana
METEPHRTLSAGEPHVSIRSAIANLQSPKYPYPICNCYYPRTGMLPPIVIANIRMICILLFPTFLMYSSDSNI

SEQ ID NO: 53 - protein, 80 aa, Arabidopsis thaliana
MIRSEKQPLLKKMLHENAGEEERDLSPRLRLRLWGFKLNLALSSMRKVKRKKSALLGGVLYSLFCPFPLFLFSHAHYYSI

SEQ ID NO: 54 - protein, 46 aa, Arabidopsis thaliana
MMSVASKKLMVMEGGGDPDTRILLSNHLNNIQTQVICFIRPYANCS

SEQ ID NO: 55 - protein, 53 aa, Arabidopsis thaliana
MRLSFSSNALIVSGLSMTSLLLIHTPFSGYHFITLQASCKLFLLILIKTTKNV

SEQ ID NO: 56 - protein, 46 aa, Arabidopsis thaliana
MKYSDVHIVVPYLQKIHSLKDLWFRMKMKGNGDRRRIDMGTEDEER

SEQ ID NO: 57 - protein, 33 aa, Arabidopsis thaliana
MQLVNCGRWTNYRTNGPISPYLVKSHKQVKSAF

SEQ ID NO: 58 - protein, 66 aa, Arabidopsis thaliana
MSAKMIRRVFSAAQRKKISLGRAGNRCAQLGGCRGVLVSKWEVDPILSSPGSIPGEDLSSNQERDG

SEQ ID NO: 59 - protein, 69 aa, Arabidopsis thaliana
MVTYSRDELVKRPSVNTNASGYPSFCSVASSSLDESGRIPFKTPNGSPLHVKVLTGEKFGPRVPYPDQF

SEQ ID NO: 60 - protein, 48 aa, Arabidopsis thaliana
MLNKKKKKYIKMTPFRWRLLGGGIHYNTLKVEHNESGPLSLKVKQLNR

SEQ ID NO: 61 - protein, 48 aa, Arabidopsis thaliana
MFLYISCISMTMYSREYEGSNNRQLWRQDDKNNLRACRVRGHREIIKQ

SEQ ID NO: 62 - protein, 65 aa, Arabidopsis thaliana
MLLRIHDRSFKVYIISAALGSSMQVQGLNKVKSGYGRWNTFWLRLLFVCSLLFQFTYHFLILQHG

SEQ ID NO: 63 - protein, 64 aa, Arabidopsis thaliana
MVLSHFTKHVPLHRYGVDFGNGKEEILKHASTFLEPQLSEALLDCLVDTQFEVKYQPYDWGLNN

SEQ ID NO: 64 - protein, 40 aa, Arabidopsis thaliana
MFSSATDPSKIKRVSVTSKRKDLEFKRGKGERILFLNSVY

SEQ ID NO: 65 - protein, 38 aa, Arabidopsis thaliana
MDLITAATVIAVFLVVIVGYIILYCIEGTNKDMNETNA

SEQ ID NO: 66 - protein, 60 aa, Arabidopsis thaliana
MIIRYVILLRPVSTWHSDLASSIGQSVAAGIANRCIKYPVSLLVVLRSINVVDRTSLSPR

SEQ ID NO: 67 - protein, 46 aa, Arabidopsis thaliana
MSMGCGEKRISFLNFMSLKVMMVAEIFPAINNTVFNLSLVARFKFN

SEQ ID NO: 68 - protein, 32 aa, Arabidopsis thaliana
MINWVYARVKSGLEKRRKLHATPLSPQHPIQL

SEQ ID NO: 69 - protein, 43 aa, Arabidopsis thaliana
MKSNPNSPNPIDLSQLLHAGLVQSEKEREGIPNLFGTSPNYRL

SEQ ID NO: 70 - protein, 24 aa, Arabidopsis thaliana
MRRCYFPTLDLTEKTFFSSLKEIV

SEQ ID NO: 71 - protein, 96 aa, Arabidopsis thaliana
MENLRNGEDNGSLIPFTFFDQSSVTIPLLKCSGLESSSSSSSSCDLSSSHSEEDESIDIKEEEEEEEEDGMTIEIKARGKNKTKPTPSSGKGGKHN

SEQ ID NO: 72 - protein, 143 aa, Arabidopsis thaliana
MATKLNLLFTILLLTLTLSHSRPARPESSSSTGSYSDQLKKHSKDNYNKGYGSGGYPGLTTEPATGFILPGSGPGGSYSELSGGYSKGRGVRLTVMCEEKGHCYMKKLTCPAKCFKSLSRKGKGYGGGGGGCTIDCKKCVAYC

SEQ ID NO: 73 - protein, 82 aa, Arabidopsis thaliana
MMSIFSPFEALYAESNGFKMIFSGTKKQSSGDNQSPATDQQKNAGKTPISSDDEKSNKKKIQPMRIAPELDGLHCFETILPF

SEQ ID NO: 74 - protein, 123 aa, Arabidopsis thaliana
MARSPYYGSSYLDYLSLPNPHLCFLFIVVFFVLSFTWYLNFESIIEDTLDHLKLVFIFTPLFLLLLVHFFSGGLSFYVPWPEQDSIHRAGSSPWGVAAVLVLILFMVSYQSDFQERWFPFGAK

SEQ ID NO: 75 - protein, 142 aa, Arabidopsis thaliana
MEMILISSSTLRFHPLRTSQRIATGTNRRRTTTVCADYYRGGRTVDENMVVLRKRIHEMKMVERNFEPPSHWMQWEKRFYCNYDATICDALTLLQTFLMNSRPSVAFGTCLLLLVSVPVSSAVFAFRILDLALWLLDAVHVV

SEQ ID NO: 76 - protein, 154 aa, Brassica rapa var. glabra
MEAIRFSNLSALLILLLLSLSLCVTSKDQTVSCTMCSSCDNPCNPVPTSSPPPPPTPPSSSGGSGGGSYYYSPPPPPSISGGGKCPPPYGGYGDGGQSYYYPPASYGNFPTPPPPNPIVPYFPFYYHIPPPGDSGSDRLLGSLLFVIFSLLLCF

SEQ ID NO: 77 - protein, 103 aa, Brassica rapa var. glabra
IVVALICAFVPAFSVEEAEAKSLWDTCLLKISPKCALDIIGVVFENLTITDACCHDLVQEGKMCHDTLIKYIAEKPHLVAHETEYLKKSDDLWTHCVSISQTA

SEQ ID NO: 78 - protein, 59 aa, Brassica rapa var. glabra
PLLRFYSSKNEEELTSLDEYIENMGENQKAIYYLATDSLKSAKSAPFLEKLIQKDIEVY

SEQ ID NO: 79 - protein, 11 aa, Brassica rapa var. glabra
LQKNGAEEGQV

SEQ ID NO: 80 - protein, 32 aa, Brassica rapa var. glabra
MSTQQEMVGECSWTGLQCRSFCGGEMNGDEAE

SEQ ID NO: 81 - protein, 23 aa, Brassica rapa var. glabra
REKENHRIKDFLGWEREGTRRHE

SEQ ID NO: 82 - protein, 95 aa, Brassica rapa var. glabra
MVPPHPHYWRKRIPLNPQPQVLLLLVICFFFVHHCDASRASSPSSVSYRNPNNDHNRNTMRRGHFLGFLPRHFPVPASGPSRKHNDIGIQALLSP

SEQ ID NO: 83 - protein, 131 aa, Brassica rapa var. glabra
MNNLFIFLILFSLCIEHTIGGEIKLVLVNELHNKVLAIRCKSKNDNLGDHDLRVGQSKRFEFSDNVWKTTLFWCNMWQAPSYRVHQRFDAYRSKWKRHLDPTYFWIAREDGVYFREDELYGYKPPVKRYDW

SEQ ID NO: 84 - protein, 159 aa, Brassica rapa var. glabra
MAQNCLNPSFPISKTTTNHSHSLKNLKNPSLNSRWRLTVNRIRCSASSDVVGNNTLRTCKHCKTQFDPVHNSPQACRFHTAHFGGTVTVFIQNLFRGMNERIIIFYLLGETKRKFESVYTGGTMDTPNSGKVLQYWHCCGSEDPFDPGCTASPHSTYDD

SEQ ID NO: 85 - protein, 126 aa, Brassica rapa var. glabra
MASNTCFSLFATIALLLLVNISGRTLPATADPTNIAARLNGGGLMECWDALYELKSCTNEIVLFFLNGETKLGDGCCHAVDVITINCWPSMLTSLGFTSEETNVLRGFCQSPNHGGSTPAPSPVKL

SEQ ID NO: 86 - protein, 69 aa, Brassica rapa var. glabra
MKRQIVLVIILVVIFLIVLDVTQVEARRMRPFPDAVDEIKLLFQALQRGPVTGSGPNGCTNIPRGSGRC

SEQ ID NO: 87 - protein, 92 aa, Brassica rapa var. glabra
MTVSKTTLLIVQVALLSCLFLSLSMYIYVCVMPNMKPGRYVQDCARASCQPKLAQPNQYFGSCFKNSDCKNSCFDSCKFQKCTNHQCDCGEC

SEQ ID NO: 88 - protein, 148 aa, Brassica rapa var. glabra
MSRRGSKRVSFSPDPEGGNEEPIIYPTHNGLTSSQHGRRRVVSDGVFSFRVRSSPRARRLLRRIGAGVAKTLRFMSLGRKTSSNTKSTQSTSTSSSSLSCSSSSSIYLVKSKSVNESESHRAEAIEDCIEFLHSSSSLSRSNSISAWS

SEQ ID NO: 89 - protein, 111 aa, Brassica rapa var. glabra
MDRYGALILILLISVSHLVSFSTARNFPGEFLPVTDGIIISGEVSALSKTVNAKLATVVGCKSEDDHFMAGYSSSPGVYAAGIYGSLVLNVLPKGSVPASGPSKRINDVKT

SEQ ID NO: 90 - protein, 37 aa, Brassica rapa var. glabra
MAHLELSIPWDGSTKQPPRPTYLKFENRSRTLRPRCL

SEQ ID NO: 91 - protein, 16 aa, Brassica rapa var. glabra
ARSSIGGQEPWSGGKT

SEQ ID NO: 92 - protein, 21 aa, Brassica rapa var. glabra
MKLILYMSFVGRNCFPGSLTW

SEQ ID NO: 93 - protein, 9 aa, Brassica rapa var. glabra
CYFFCYVLP

SEQ ID NO: 94 - protein, 39 aa, Brassica rapa var. glabra
LVRKTHCRKDGTFLDERAEALVLEVEQAVEEMLQDGSPL

SEQ ID NO: 95 - protein, 34 aa, Brassica rapa var. glabra
TVSTNSPTHSSQTDSHVSNTRSRRQTKAPAYLDQ

SEQ ID NO: 96 - protein, 27 aa, Brassica rapa var. glabra
YLDLSSSEPVCKYDSQLVPRSRWRRRG

SEQ ID NO: 97 - protein, 20 aa, Brassica rapa var. glabra
LLFLIVPICSLFRDSLICII

SEQ ID NO: 98 - protein, 14 aa, Brassica rapa var. glabra
IYRGSNIFILYTLE

SEQ ID NO: 99 - protein, 59 aa, Brassica rapa var. glabra
RRRRKPKNLKPNQGSSPLSSPARLLRPEKALAFRVSSPITTLSGERSLTQNLMSITTKR

SEQ ID NO: 100 - protein, 36 aa, Brassica rapa var. glabra
MTTVNMATREGRTKLKETGKAKGSGNTRQHRWLHLK

SEQ ID NO: 101 - protein, 46 aa, Brassica rapa var. glabra
YQSFPVHWLLSSSPSNSLPTPLPPPQLSLYLNPCSSESLSFPSQHS

SEQ ID NO: 102 - protein, 26 aa, Brassica rapa var. glabra
MGRLIFRPISLGFFSADYSSRVGIRE

SEQ ID NO: 103 - protein, 22 aa, Brassica rapa var. glabra
SLIVNCWTIDFATSHKPVILSH

SEQ ID NO: 104 - protein, 15 aa, Brassica rapa var. glabra
GIWWWWWLRSSVEMD

SEQ ID NO: 105 - protein, 43 aa, Brassica rapa var. glabra
TPVLWVLRTLCHPLQQSRFMSGGTSMKQDLDKLVKTKEGRSFF

SEQ ID NO: 106 - protein, 33 aa, Brassica rapa var. glabra
DVEIQYGARYEGTRMKGRIGTNGYWGATEKNNT

SEQ ID NO: 107 - protein, 35 aa, Brassica rapa var. glabra
HSRGVRLITNREKEGGERYLIPVRKRGVEETERRR

SEQ ID NO: 108 - protein, 55 aa, Brassica rapa var. glabra
MKPKLGELTKRRQSKGQLRAAAETLYQNGEAVNITRNPPHFHLPMERNHNIGPFH

SEQ ID NO: 109 - protein, 42 aa, Brassica rapa var. glabra
KACCNKEQEACNSSCNVCNQRSPFEDLYKMDADQELLPYCLR

SEQ ID NO: 110 - protein, 47 aa, Brassica rapa var. glabra
VESKEKNEERDERDPSGSGTRADAPAVRRRVRFTSLFQHINEVHMLL

SEQ ID NO: 111 - protein, 22 aa, Brassica rapa var. glabra
DGCHSFIVDSATLAFLYSPTSW

SEQ ID NO: 112 - protein, 35 aa, Brassica rapa var. glabra
DLRVDTSEGGTKAWGHVLPIHKGKGVDMLLSYVIK

SEQ ID NO: 113 - protein, 26 aa, Brassica rapa var. glabra
GPHINLQIALRTRTNKCHVKSYKTAT

SEQ ID NO: 114 - protein, 26 aa, Brassica rapa var. glabra
LTITIQRIIHSINENFHVRLLYAIFF

SEQ ID NO: 115 - protein, 38 aa, Brassica rapa var. glabra
GVPDPVRDPFPSSRPQPVSETRSQPARRDPIAACSRSR

SEQ ID NO: 116 - protein, 26 aa, Brassica rapa var. glabra
LSPYLNCHSHHYHQHSEPWSHCWLKP

SEQ ID NO: 117 - protein, 20 aa, Brassica rapa var. glabra
RRLGEMGSLYGLDCEKILLC

SEQ ID NO: 118 - protein, 11 aa, Brassica rapa var. glabra
TFGAKVALESS

SEQ ID NO: 119 - protein, 39 aa, Brassica rapa var. glabra
SVLALTIVLHFQGRCHKERSGFEGPWTQNPLIFDNSYFK

SEQ ID NO: 120 - protein, 44 aa, Brassica rapa var. glabra
PPYPHPPSQHPRTWHLPPLYSPHIFHLCIHIRHHFLLLTSSTSV

SEQ ID NO: 121 - protein, 32 aa, Brassica rapa var. glabra
QRILKKKKPSSVGEEEEEEAPTLELCFGFHLN

SEQ ID NO: 122 - protein, 42 aa, Brassica rapa var. glabra
QIKFTSIVYYKSASKHIKSIIAHLTSSPVHFLTINSSCKTFL

SEQ ID NO: 123 - protein, 21 aa, Brassica rapa var. glabra
ISSVQDFWFGMSRRGGGRRRR

SEQ ID NO: 124 - protein, 45 aa, Brassica rapa var. glabra
RQLFSLGSWYSQTAPVGYGCQGTYRSVWQVDVQLQGGPGQELSQN

SEQ ID NO: 125 - protein, 16 aa, Brassica rapa var. glabra
RPTLRTSYPLYPSYCS

SEQ ID NO: 126 - protein, 35 aa, Brassica rapa var. glabra
INKKKKKTFRWTSNRWNRKGNSNIYLVSKYELNPS

SEQ ID NO: 127 - protein, 40 aa, Brassica rapa var. glabra
FIYCLSLSLFSLVHQFADCNGRRRWRHWEENKFRWWRWRG

SEQ ID NO: 128 - protein, 28 aa, Brassica rapa var. glabra
LQIHDRLMLVHMVSIDIGCMSSIDTLNE

SEQ ID NO: 129 - protein, 53 aa, Brassica rapa var. glabra
LYRYAVDFGNGEKSVLKHASTYLEPQISEELLDVLVDTQFRVVYQTYDWGLNH

SEQ ID NO: 130 - protein, 20 aa, Brassica rapa var. glabra
SASDPSKTKRSKATEAQPDT

SEQ ID NO: 131 - protein, 39 aa, Brassica rapa var. glabra
STPSSPSPNDVDLSTLVVSGLLRSTDSTLSASSLFPAKP

SEQ ID NO: 132 - protein, 19 aa, Brassica rapa var. glabra
FPTLDIPEETFLSSLKELV

SEQ ID NO: 133 - protein, 95 aa, Brassica rapa var. glabra
MENLRNGEKGHSHWIQFMLFDQSSITTLLLKCLGLESSSSSSSSSSVSSHCEEEDDIQMKEDEDDMTVETTERRLQKSKPKPKPKPSPGIGGKIN

SEQ ID NO: 134 - protein, 155 aa, Brassica rapa var. glabra
MTTKLIVLFTLLLLTLTLSHSRPTGPESVSDQLKKSSKGNNNKNDKGYGSGGYFGPGTGSVPGTGFGLPGFTGKDWGNIGGGYGGGYGGPTGGYNKGGVVRPTVVCKEKGHCYMKKLRCPAKCFKSFSRSGKGYGGGGGGGGCTIDCKKKCMAYC

SEQ ID NO: 135 - protein, 84 aa, Brassica rapa var. glabra
MMSIFSPFEALYAESNGFKMKLPGGQKQSSGDSQSPATEQQKNYGRIPITSDDEKNKKKEKKIQPMRIAPELDGVHCFETILPF

SEQ ID NO: 136 - protein, 123 aa, Brassica rapa var. glabra
MARSPYYGSSYLDYLSLPNPHLCFFFMVVFFVFSFTWYLNYESILEDTMNNLKFFIILSPLFLLLLVHFFSGGLSLYVPLPEQDSIHHAGSSPWGVAAVLVVILFMVSYQSDIHEMWFPFGAK

SEQ ID NO: 137 - protein, 142 aa, Brassica rapa var. glabra
MERTLISSSPFRFHPLTTSHRIATITKRRKQTTVCCDYYYQGGRVVDENMVVLRKRIHEMKMVERNYEPPSHWMQWEKRFYCSYDATICDALCILQTFLMNSRPSVAFGTLFLIFVSVPVSTAFFAFRIFDILLWLMSAIHV

SEQ ID NO: 138 - protein, 127 aa, Glycine max
VMFLSLNALAIALEDHNLKTTSKDDIKCTPCGQIPSPPPPSPPPPAPTTTYCPPPPSPPSSGGGGAYYYSPPPPSQYTYSSPPPPASTSGGGGGGAYYYPPPRGYYPTPPPPNPIVPYFPFYYHTPP

SEQ ID NO: 139 - protein, 73 aa, Glycine max
CGSHLYPNCGDEVFSAIFFGNKTVSEYCCDKLVNDVGKMCHDDMTKYILTLPKFRAHETQILKRSQTVWFDCV

SEQ ID NO: 140 - protein, 61 aa, Glycine max
PLLRFYTSKSEEELKSLDDYVENMGENQKAIFYLATDSLKSAKTAPFLEKLVQKDIEVWLL

SEQ ID NO: 141 - protein, 17 aa, Glycine max
WSLCFSLWVVARVLLHC

SEQ ID NO: 142 - protein, 82 aa, Glycine max
YCNNNMVLHRRPLILVIIWLFLFLFILGHCHGSRATTTVFKFKPKSPHHGHFSGFLPKRMPIPYSTPSRKHNDIGLRSWRSP

SEQ ID NO: 143 - protein, 20 aa, Glycine max
SCFFMTDVIKGARNSNSLCD

SEQ ID NO: 144 - protein, 128 aa, Glycine max
IIVFLIIVCVQDGLSDDAFYGGTTHVRVYNGMNDSIPVHLHCRSKDDDLGQHVLAVGEHQEWSFGTRVFGNTLFWCTMDAGNVHASFEIYNAKTEVATCNAQCNRILNNDGGYFFNEYSGYWEKRLSW

SEQ ID NO: 145 - protein, 51 aa, Glycine max
GETKRKFESVYEGGTMNTPDSGKVFQYWHCCGSEDPFDPGCTASPHSSYDD

SEQ ID NO: 146 - protein, 110 aa, Glycine max
VTITALVSSMVESRSLPNASSSLTVRLKLEGEPSNCWDSLWQLQACSGEIVTFFVNGETYLGHGCCQAIRVIGHDCWPNIVASLGFTNEETDVLEGYCDQAVDSPSPPSI

SEQ ID NO: 147 - protein, 63 aa, Glycine max
RVLNIILVLFVLLTIVHVKQNMAARVLNMENIPLRLQSLNKGPVPPSGPSGCTYIPGSGSTHC

SEQ ID NO: 148 - protein, 17 aa, Glycine max
GHLFRSSDCQNSCCFFC

SEQ ID NO: 149 - protein, 124 aa, Glycine max
MMLRTTEKVCYTPDFNEKPIMFLKQTSVGGGGNKKRVTGTWTFRFPKDPKFSPVRFLQQLGAKVASAIRVVSMRRRSSRKVSSSSLVRTRSVSDPPDSHRAKAVEDCIEFLHSSSSRERPSSVS

SEQ ID NO: 150 - protein, 28 aa, Glycine max
GKYGPMILNMLPKGPVPPSGPSKGTNNL

SEQ ID NO: 151 - protein, 37 aa, Glycine max
MAHLELSIPWRGSTEQPHRPTYLKFENRSRALRPRCL

SEQ ID NO: 152 - protein, 50 aa, Glycine max
NGFCPVGPLRDRGGSSSKEVDRAQKKLSKATFQPVHLARGLGGLNPWVGG

SEQ ID NO: 153 - protein, 61 aa, Glycine max
YSDETVLKILNVKLNCQIKYDKGYKKKYILTYLNYTSSQKKKTSPNFLIILNSFLFIKYYF

SEQ ID NO: 154 - protein, 56 aa, Glycine max
IIIYLKGIYKKKNESKNIREYKDTPIRIQRSKEYRTIIFNGAHRTRTNMKIRCIFI

SEQ ID NO: 155 - protein, 37 aa, Glycine max
HEHRRFAYVLYRNTNLFAWHPFDMPRIQPSIICHKLA

SEQ ID NO: 156 - protein, 35 aa, Glycine max
FMIPSLKFVNIVLAECLMFSLHTIIMIGKRECILW

SEQ ID NO: 157 - protein, 29 aa, Glycine max
STFEYFSKLMTICFVWFMCICLFACVYQC

SEQ ID NO: 158 - protein, 22 aa, Glycine max
NLHHPLDYKYFSTPPKIFSPSP

SEQ ID NO: 159 - protein, 58 aa, Glycine max
ILKGRREMPTPSLRRPHGSNPHRHLMVHISTRNIIRASQLTPGTPLIQHPCSLRHPSR

SEQ ID NO: 160 - protein, 49 aa, Glycine max
WTLLTVHRFVCIFNTHTHIHSHTPTHTHSHTHTHTQKHTHTHTETHTHK

SEQ ID NO: 161 - protein, 23 aa, Glycine max
IWRFATEASKHREGIKQARNLRR

SEQ ID NO: 162 - protein, 25 aa, Glycine max
IMHKIVVELCNKIFQHVQKMLVHCW

SEQ ID NO: 163 - protein, 47 aa, Glycine max
KQKYFLNVENCKWKIFKKKQNKDKNDKSKITIFVSTPYKFRKMNFKK

SEQ ID NO: 164 - protein, 25 aa, Glycine max
HPLLYATWNNSLNHHFNPIKSPTIF

SEQ ID NO: 165 - protein, 37 aa, Glycine max
RKSQVHLTKVAETVNEYAQSEKVWLNSSEFHLLVKRN

SEQ ID NO: 166 - protein, 20 aa, Glycine max
CNKEQKAYSSSCSVYNRRLV

SEQ ID NO: 167 - protein, 29 aa, Glycine max
VSLRYCFLGPGTFTYILQNLTLLWLFKPT

SEQ ID NO: 168 - protein, 27 aa, Glycine max
KLKFERDRLCDVCQKGKQVRGYLMVIH

SEQ ID NO: 169 - protein, 29 aa, Glycine max
RNVSSLKKFRKEKYLLHIQSNERYNCQRK

SEQ ID NO: 170 - protein, 15 aa, Glycine max
KGTLHPGSWEFKEWR

SEQ ID NO: 171 - protein, 51 aa, Glycine max
MCTCPKARLFSMCEYSVESVYLEMNSHFLCIFILSGERATISKNLLMHLIN

SEQ ID NO: 172 - protein, 41 aa, Glycine max
VVHTHFPRGVLIPSLCVNNSRTLIFEICIPLFGFFLMFSSN

SEQ ID NO: 173 - protein, 19 aa, Glycine max
SLILLSNFCPIFLYSHCHY

SEQ ID NO: 174 - protein, 46 aa, Glycine max
VHLSFWSSPLPTHHLSPSHYLLVHSPFRIYHLPHLSLSLSLTSLSL

SEQ ID NO: 175 - protein, 33 aa, Glycine max
SRDRNIKSPSHNSGASNIKVFESIGGNSVEESG

SEQ ID NO: 176 - protein, 33 aa, Glycine max
WFWYWLRQLFFLILNGTFICSILFSNCFHFIII

SEQ ID NO: 177 - protein, 50 aa, Glycine max
RYSIDFGKNEVEVIKHVSNYLDSADSEVLLDLLATSELKVTYQPYDWGLN

SEQ ID NO: 178 - protein, 44 aa, Glycine max
VLPLPLFTWLETMPSSLLANASSGLLYRCLNHLHQLLVIFFSIN

SEQ ID NO: 179 - protein, 79 aa, Glycine max
FTFITQEEEERPLIRKRKSPETSTKSADQADVGNSQAQMPKKKKKVKQVEPEPSVTVEGGEPARKKKKKTKSSKKQGHH

SEQ ID NO: 180 - protein, 149 aa, Glycine max
KMKPTITILLLSLLLLITSPSLATRPASNPDQVKHNKNNNQGGGAGAGAGGFFGPGGGFSIPGFGNGFGNGIIGGGYGSGYGGPNGGSSKGGIIRPTVVCKDKGPCFQKKVTCPAKCFSSFSRSGKGYGGGGGGGGCTIDCKKKCIAYC

SEQ ID NO: 181 - protein, 80 aa, Glycine max
MMSIFSHLEIAQAQNWRFSLGTGPVKGVNSKPKMEATSSSAARKDPNPSSTAGSKQNPPRLRPRFAPELDGLHCFESIVP

SEQ ID NO: 182 - protein, 118 aa, Glycine max
PYYYYLWEYFSFPLHLIFFVLILFFVLAFSWYINYESLLEDLLVQVKIFLALVPLLLLLLVHCLSSGASFPIPLPEERESLHRAGGSPWGVALLLLFVLFMMAYQSSFHQRWFPLATR

SEQ ID NO: 183 - protein, 143 aa, Glycine max
LVNSSPILFHPFSPSKPCSKGKKTNSFAPIKGSMRGSADQNFSGRLVDEGMIILRKRIHEMNMIERNYEPPSDWMDWEKRYYTKYDSIICEAVGVLQTQLMNTRPSLALGAMALVAISVPTSSAVLFFHLFELAKAVLAAAHL

SEQ ID NO: 184 - protein, 134 aa, Solanum lycopersicum
IECTMCAACDNPCATPSPPPPQPPSPPPPPSTSYNCPPPPSPPSSGGNYYYSPPPPPPSSSGGGGGGNYYYSPPPPYQNYPSGPTPPPPNPIVPYFPFYFYNPPPPSQSAAIKLTDFSPKSSIFFTVFIALLIF

SEQ ID NO: 185 - protein, 78 aa, Solanum lycopersicum
CLQNISDDCGVIIFNKIFDNGKPTDVDECCGQLLTLGKKCHHTIIESTIHSPEMKGVNKTAVWINSENLWKRCVFISR

SEQ ID NO: 186 - protein, 53 aa, Solanum lycopersicum
PLLRFFSSKSEEELISLDDYVENMGENQKAIYYLATDSLQSARTAPFLEKLVQ

SEQ ID NO: 187 - protein, 30 aa, Solanum lycopersicum
MEREEEERRLWRREARRKEWGERERRLERG

SEQ ID NO: 188 - protein, 74 aa, Solanum lycopersicum
FFRRKILVLWMAIILISIFGHFCHGSRSNSQVFNTINNQRNSYNHGHFWNLLPKRIPIPASGPSRKHNDIGLKS

SEQ ID NO: 189 - protein, 141 aa, Solanum lycopersicum
SLFSFIFLLNLILRSCGAPFSSIRVVYIIDALESSSKPLTVHCQSKDDNLGYKNLHSGEEYNFRFQEKFFGGTLFFCHFWWDDKNIIFDVYNDDIATFCGDLIPLEGFDYECFWKVQEDGFYFAPHRDPPSGYEKKHDWSK

SEQ ID NO: 190 - protein, 51 aa, Solanum lycopersicum
GETRRKFESVYSGGTMDTPDGGKVFQYWHCCGSEDPFDVGCTAAPHASYDD

SEQ ID NO: 191 - protein, 113 aa, Solanum lycopersicum
IILLVSWLLTTTTIATNMSKIKPGQGIAARLQLLEGGDGGGASMPVCWNALFELKSCTNEIVLFFFNGESYLGKDCCKAIRTITYNCWPSMLSSVGFTAEEVDVLRGYCGTPS

SEQ ID NO: 192 - protein, 20 aa, Solanum lycopersicum
QSLQRGPVPSSGPSGCTNIP

SEQ ID NO: 193 - protein, 47 aa, Solanum lycopersicum
CECQCQPQHECEHQHQCQRKHECQCQCKCKCHWKCQRKCQHKHKCEC

SEQ ID NO: 194 - protein, 132 aa, Solanum lycopersicum
KRMSRRVNFSPDNFHEQNNNPSIFYFKKKPLCSSNWNSRLQKSYSSSSYVASFVKNIGIKVASALNFLSNTSRKKRSSCKVSSSSTTTSLTRSSSYAQTSIYDSQRAQAIEDCIEFLNSSSSSLPRSSSVSS

SEQ ID NO: 195 - protein, 25 aa, Solanum lycopersicum
NMLLNRLPKGKKPVSGPSKRTNNLK

SEQ ID NO: 196 - protein, 37 aa, Solanum lycopersicum
MAHLELLIPWRGSTKQPRRPTYLKFENRSRALHPRGL

SEQ ID NO: 197 - protein, 18 aa, Solanum lycopersicum
CSLDYRRIRGAVLGNDLT

SEQ ID NO: 198 - protein, 37 aa, Solanum lycopersicum
ILLQAEQHFLKHNDAGSWIQDSALMLSMCKEVPWYLV

SEQ ID NO: 199 - protein, 31 aa, Solanum lycopersicum
FQNFIFSPLPPAPTPTTAPSLFLKNIFDFKI

SEQ ID NO: 200 - protein, 31 aa, Solanum lycopersicum
LVLDFSSPNYSCIHAYKKLSSPFGSWYLDIL

SEQ ID NO: 201 - protein, 37 aa, Solanum lycopersicum
RMEGITKIWARNLQKFVEARGEYQTTRYPTRKPLNTS

SEQ ID NO: 202 - protein, 44 aa, Solanum lycopersicum
RAALSDQGPGHKGPGTIQVRRTPEVTAMSRNLTHGPKRRANTRT

SEQ ID NO: 203 - protein, 29 aa, Solanum lycopersicum
FRILIGICVWIFSIPSQSRFVRLHSFSNL

SEQ ID NO: 204 - protein, 20 aa, Solanum lycopersicum
HPRMASYHCWCYGLSSWSRF

SEQ ID NO: 205 - protein, 35 aa, Solanum lycopersicum
VLFKLTKYELDMMNRESPPVKPSIKKSPKLELKPL

SEQ ID NO: 206 - protein, 20 aa, Solanum lycopersicum
NSTLYHPINLYTWHTAIQHH

SEQ ID NO: 207 - protein, 35 aa, Solanum lycopersicum
ERERRSTIDDEKERGGRRSTTRERGAPSTRSVRKR

SEQ ID NO: 208 - protein, 32 aa, Solanum lycopersicum
LNGLSMQYNPEQVHLIVEHGQTILFFHKVDNT

SEQ ID NO: 209 - protein, 21 aa, Solanum lycopersicum
KASCSMEQEACSSSCNDCNQK

SEQ ID NO: 210 - protein, 22 aa, Solanum lycopersicum
IKGCTFIFYFRPSSWTYLSRDY

SEQ ID NO: 211 - protein, 36 aa, Solanum lycopersicum
ISVMILPRMILHHLNMNMDQAELFLVWLLDFHPLCF

SEQ ID NO: 212 - protein, 28 aa, Solanum lycopersicum
PDNSPKNTTRSSPKWTTPTYYTENYEQS

SEQ ID NO: 213 - protein, 39 aa, Solanum lycopersicum
TSTFQGTYSSRHVRSWTFDLHTSPNFMNFLKTSLITILT

SEQ ID NO: 214 - protein, 27 aa, Solanum lycopersicum
FAVWGMSKLFLCFNMMRLRLNISYFFI

SEQ ID NO: 215 - protein, 47 aa, Solanum lycopersicum
ILEKVSGPLNLATSLGFIMNWPMSWLFWHQCFQILESLTSLHWRFKF

SEQ ID NO: 216 - protein, 50 aa, Solanum lycopersicum
SILKALINISSIKRPYIVNDIYIVLPSTLPPFHIKKKVLLCDLKLNIYKM

SEQ ID NO: 217 - protein, 33 aa, Solanum lycopersicum
KNERNLKRKIKVGLWGFEPTSSLTRRREVKEKK

SEQ ID NO: 218 - protein, 44 aa, Solanum lycopersicum
IFSERKMTSLIYKRKYFSYNHFFILPVSHNIYTLLVLISSTQNI

SEQ ID NO: 219 - protein, 34 aa, Solanum lycopersicum
CSYSSSSLGEASKTPLSEPMRSPLVEYIHGEEYG

SEQ ID NO: 220 - protein, 49 aa, Solanum lycopersicum
YEINILIFSLGSSLQVQFLNKLGLSYLLINRNWLTPLSQYSLIYPILHN

SEQ ID NO: 221 - protein, 51 aa, Solanum lycopersicum
RFSVDYGNNEVEVLKHAANYLDSSVSQAMLELLANGQLKVVYLPYDWGLNN

SEQ ID NO: 222 - protein, 44 aa, Solanum lycopersicum
IIIIRSHHKSSSSSSSSSSSSSSSSSSSSSSSEEEEEEEEEEEE

SEQ ID NO: 223 - protein, 143 aa, Solanum lycopersicum
KISITLLALLLIITFNSTIATRHIHPNKHGVGKTKKGSGEVRNNNGGDVGFGGIFGPGGGFNIPGLGGGVFGGGFGGPKGGYGKGGVIRSSVVCKVKGPCFGMKLICPAKCYKSYSSAGKGYGFGGGSGGCTMDCKKKCLAYC

SEQ ID NO: 224 - protein, 89 aa, Solanum lycopersicum
IMSIFSSFDALSAEIFGLKVTPSWAPATSDNKQQQGLLSHRKTAASPTSNSSTAELNKAGEAAPAQQRRRPRFAPELDGLHCFETILPY

SEQ ID NO: 225 - protein, 105 aa, Solanum lycopersicum
PSIHLCFFFLVLFMFVSVTWYINYESIFEGIMDQFKFLLMLSPLVLLLIVHLLSHFEKSLMFNIPLPEQDSFHRAGGTPWGVGFLLVLLLFMISYHSDLRERWFP

SEQ ID NO: 226 - protein, 136 aa, Solanum lycopersicum
STSFLSSTPFSSRRKSSSSSRNSRTTALAMGREAQDRNYYNCRSVDENLIILRKRIHEMKMIERNYEPPSDWMDWEKSIYANYDANICQALGLLQSQLMDTRPSLVLGMAALIGLSVPISTIVILFHLIELTKGIL

SEQ ID NO: 227 - protein, 133 aa, Oryza sativa
IVLKDGTTCKLCASCDNPCNPSYYPPPPPPVVTPTPQCPPPPSYPSGGGGGGGGGTVMYTSPPPPYSGGGGGSSTGGGGIYYPPPTGGGGGGGGGWQQGGGGGGAYPTPPPPNPFLPYFPFYYYSPPPPFYSS

SEQ ID NO: 228 - protein, 47 aa, Oryza sativa
LQYASKCCHIESTKYISSQREESGQKSHNMKKIRQNMWSDIIQTGKT

SEQ ID NO: 229 - protein, 63 aa, Oryza sativa
PLLRFFSSQSNEELISLDEYVENMKPEQKDIYYIAADSLSSAKHAPFLEKLNEKEYEVCTVNS

SEQ ID NO: 230 - protein, 30 aa, Oryza sativa
TWGKMCTYLLGKAKLGSLPTPCRDDWVITR

SEQ ID NO: 231 - protein, 35 aa, Oryza sativa
RRREKHLGVNLGWKKEGWKKKGGEEERGFSRGDGS

SEQ ID NO: 232 - protein, 62 aa, Oryza sativa
LLLLLLLLMVPRCKASRGMQTFKARPLERGASNHFLGFLPRGPIPPSGPSRQHNSIGLDSQL

SEQ ID NO: 233 - protein, 46 aa, Oryza sativa
GARRTQKEEEEGEKERGEGRGRRKDEDDDMWGPTIFMCELHVGPTY

SEQ ID NO: 234 - protein, 53 aa, Oryza sativa
YPGETKRKFESVYAGGTLDTPDSGKVFQYWHCCGSEDPFDVGCTASPHCSYDD

SEQ ID NO: 235 - protein, 131 aa, Oryza sativa
LLSVAVVLVVVSAQALAAVAVADAARVNAGAAAFSPAVPLGGRLDGGGGGLVECWSAVAELRSCTDEIVLFFLNGETTQLGAGCCRAVRAATRDCWPAMLAAVGFTAEEADVLRGLCDAEAAAAAADSTSP

SEQ ID NO: 236 - protein, 51 aa, Oryza sativa
RMVESMRSFLRVLTCRADDVARADAAVQRPPVPTGPRPAAHVPRPTPPPHG

SEQ ID NO: 237 - protein, 37 aa, Oryza sativa
LTICGKCSTTSDDTASCSIQRSARSEYHRASCSVSSS

SEQ ID NO: 238 - protein, 74 aa, Oryza sativa
TATRFYRRARASVARAFRPASTKKGPASCAASRSPDCTPARNSSRRHSLAPVVADDSHKSEAVEECIRFMNSSS

SEQ ID NO: 239 - protein, 32 aa, Oryza sativa
YASGVVDKYAPLLLSMLPRGPVTPSGPSGGTN

SEQ ID NO: 240 - protein, 53 aa, Oryza sativa
GVDWPRQSGDWLGRHRAEVGQRAAALDWPRRLRCKAPGRGRGGVGFVQPWHSG

SEQ ID NO: 241 - protein, 36 aa, Oryza sativa
DAGSWIQDSAVMLSVSKEVPWYLVINCCSCYILIFF

SEQ ID NO: 242 - protein, 26 aa, Oryza sativa
SFSLLKFLCFFYGSIKRSFLCFSCVL

SEQ ID NO: 243 - protein, 61 aa, Oryza sativa
NRNTLFICWRFKCLTTDPILYWYCVLSFPGQYWKTIATTSPSSRRHSSGKSTLRIRVEKLR

SEQ ID NO: 244 - protein, 21 aa, Oryza sativa
WKVRNRGETKHDNTLLIDQFL

SEQ ID NO: 245 - protein, 32 aa, Oryza sativa
FIKKLNSHPTCSSTCELSVVGERDKRCSIWKL

SEQ ID NO: 246 - protein, 32 aa, Oryza sativa
PCDFLTMPCQHQRCFLIFWVGLLSHHCPIKEK

SEQ ID NO: 247 - protein, 49 aa, Oryza sativa
PFDYSKLTIPPHNFVSVPFGRAPQFDGTHYAAWKHKMKLHLISLHPSIW

SEQ ID NO: 248 - protein, 34 aa, Oryza sativa
NETSRPRLSLKSFRKNRKIGKTKSPPPPHSHVYT

SEQ ID NO: 249 - protein, 24 aa, Oryza sativa
GTTQVRRTLEVTAMSKNVTHRPRK

SEQ ID NO: 250 - protein, 47 aa, Oryza sativa
FTVEFYLSLDPNEFAKLAVYIPSGCRGRFNPFSKKNEFAKYCHADRL

SEQ ID NO: 251 - protein, 33 aa, Oryza sativa
TSKHAPQLCPLLNSAVLSAAVLVPYPALYRLPY

SEQ ID NO: 252 - protein, 40 aa, Oryza sativa
ILVISSERVCPPFFKHSLILAIATWTTHVFVVSSSPILLR

SEQ ID NO: 253 - protein, 21 aa, Oryza sativa
VDGARWWWWRSGAARGDGDDD

SEQ ID NO: 254 - protein, 13 aa, Oryza sativa
SGLWGGGGFTNRD

SEQ ID NO: 255 - protein, 27 aa, Oryza sativa
RFGSNGWDATESATTQRNPLFDYTYIN

SEQ ID NO: 256 - protein, 33 aa, Oryza sativa
RRRGVQDQDEGGDAVRPQRAQAGLPRRRRRRRR

SEQ ID NO: 257 - protein, 40 aa, Oryza sativa
KTRFGQLIGCNSSKGPTTIFCQLCHRNLETAQHQFNDCTF

SEQ ID NO: 258 - protein, 42 aa, Oryza sativa
CHIICREKYESCHTICQQRSICRKRSVCQEKLSVGKSKKTLP

SEQ ID NO: 259 - protein, 24 aa, Oryza sativa
TRGVRRCHDTSTFWYSICVKLILE

SEQ ID NO: 260 - protein, 28 aa, Oryza sativa
FYGNSDTCKIYVLDDALMSFFFKKKSPT

SEQ ID NO: 261 - protein, 18 aa, Oryza sativa
ARGHRLPVHRQSSFNLHI

SEQ ID NO: 262 - protein, 41 aa, Oryza sativa
WLHHFFLKFGWVDLQTKHAPLNLEISSTKRSYCHMYKRKVS

SEQ ID NO: 263 - protein, 29 aa, Oryza sativa
ACLFRHQLQRNWFSGRAWYLFFYVEDSKY

SEQ ID NO: 264 - protein, 30 aa, Oryza sativa
PHSLAAGQPIANRPVQGHSQHPAKLSQYQP

SEQ ID NO: 265 - protein, 21 aa, Oryza sativa
THVDSTCQHDPRHHHPSPSSL

SEQ ID NO: 266 - protein, 28 aa, Oryza sativa
FKNTHSQNLFLNLNFFQRHPNLGNFWLY

SEQ ID NO: 267 - protein, 24 aa, Oryza sativa
GDELSLSPSFGFASSYRCGRNFAD

SEQ ID NO: 268 - protein, 34 aa, Oryza sativa
KITPIFIALPDAISIYSIYPIIHQLMFTSFVLHS

SEQ ID NO: 269 - protein, 21 aa, Oryza sativa
LSFCKICLGSVSVLFWFIPSY

SEQ ID NO: 270 - protein, 27 aa, Oryza sativa
YYPTKILVTLIVANVRLVCYQILPTIV

SEQ ID NO: 271 - protein, 43 aa, Oryza sativa
SPTSRSRLLSSLLPAFKANQQKGGGHFFSLVFPFPLFMFMHLH

SEQ ID NO: 272 - protein, 38 aa, Oryza sativa
IIIGILFKVLFIFLRCPINHYHSTSLRAYCLLFLLLFV

SEQ ID NO: 273 - protein, 31 aa, Oryza sativa
AALCRRRSAAACEKERGLGRGGEREARIGGC

SEQ ID NO: 274 - protein, 56 aa, Oryza sativa
PSQVMASQAFPSCCPVAANVVAGAHRIPLSELLHRPPLERMVVLAGDEHHPTLPFP

SEQ ID NO: 275 - protein, 45 aa, Oryza sativa
GRAMAAALLQCACRGYWQWDFLWLRLFAVWAFLFLLLKHMYLLIH

SEQ ID NO: 276 - protein, 50 aa, Oryza sativa
RYSTDFGKNETEVLKHAANYLEPAESEQFLELLANTQLKVLYQPYDWSLN

SEQ ID NO: 277 - protein, 30 aa, Oryza sativa
SSATDKGETRRVGAGMRGSDREGERGTGER

SEQ ID NO: 278 - protein, 40 aa, Oryza sativa
SPLRRSLPHGVHSPCVISYPISLLGILNRLNLNSSMSLVP

SEQ ID NO: 279 - protein, 22 aa, Oryza sativa
VSWVGSKASDGLEKREEYHLTP

SEQ ID NO: 280 - protein, 37 aa, Oryza sativa
EEEEEEEEEEEEESEEEQGMDMEEKGRGADNSSENPS

SEQ ID NO: 281 - protein, 164 aa, Oryza sativa
MASTARVAAALFLLLLGLSATAPLAARDLMSAAPSAKKQPAGRKPSVQPGYPGTNPGGGGGGGIPTIPGFGSIPGMGGGMGGFNVPGMGGGWGGGYGTPSGGYSRGGVVVPTVVCSDKGPCYRKKVTCPKKCFSSYSSSGKGYGGGGGGGGCTIDCKTKCTAYC

SEQ ID NO: 282 - protein, 60 aa, Oryza sativa
SAGKQQKAAAEKAPTRQQEGEKQQAGRSSPAAAAERKPVARPRFAPEFDGINCFETIVPF

SEQ ID NO: 283 - protein, 126 aa, Oryza sativa
SPAAAAGYVQAPELPLHLCFFLVVLLVFLGFSWYTSYGSAAERFADQARLLLMASPLALLLAVRLLSGGGDGERRGVDQLRQLSLPMPERDSIHRAGGSPWGVGVLLALLVVMVSYQSNFRDRWFP

SEQ ID NO: 284 - protein, 134 aa, Oryza sativa
MAMSSSMSLLTSPAELGRRGPALKARRSSPAACCAFRRDQYSGGALVDSSMAVLRRRMREARMAENNYEAPAGWSAWEKRYYPAYVSDVSAAVGALQLLLMGTRPSVAIAAAALLFAGVPVSAVAAVHHLAQLA

SEQ ID NO: 285 - protein, 93 aa, Zea mays
CPPPPSSYPPPPSSSYNTPPSSTWSYPPPPPSGGYIPYLNPPARGGAGYGYPAPPPPNPILPWYPWYYKTPPPPPSAAARGTGSSVLGLAAVL

SEQ ID NO: 286 - protein, 53 aa, Zea mays
PLLRFYSSKNETDLISLDQYVENMPENQKAIYYIAIDSLQSAKTAPFLEKLVQ

SEQ ID NO: 287 - protein, 56 aa, Zea mays
MISVIIILFFHVCHDDVFSSHAMFATSSSYSHGKNRPKRNHVVSHVPRKVSTGPTA

SEQ ID NO: 288 - protein, 39 aa, Zea mays
FWAALHRLPNFIVPVGFEPWKRLWKSWVPPRCKFFLWLA

SEQ ID NO: 289 - protein, 51 aa, Zea mays
GETKRKFESVYAGGTMDTPNSGKVFQYWHCCGSEDPFDVGCTASPHCSYDD

SEQ ID NO: 290 - protein, 60 aa, Zea mays
AERLEGAETQQCWEALVEIKSCTGEIIILFIRGEAFLGPGCCRAIRVIEQSCWAADSMMS

SEQ ID NO: 291 - protein, 44 aa, Zea mays
DCSKGRSQPASLAALFRSSPKECSRNQLFPHCRFQHQDHDVCEC

SEQ ID NO: 292 - protein, 99 aa, Zea mays
PAASVSLLERIRDVVLRLIMLSAVSRASTQPTSSSGSGKSAKASSPGDASQHRESDTCVAVAATCRREDSIRNEAVEDCIEFLKRSSAEGDAAKLSTVS

SEQ ID NO: 293 - protein, 21 aa, Zea mays
QGLLTYLKFENRLRMFHPQGL

SEQ ID NO: 294 - protein, 52 aa, Zea mays
RSASPALCAVSRRIRGAGGGSGWRQIRRRLRPLPRRPRRMREAERGCAPWSG

SEQ ID NO: 295 - protein, 39 aa, Zea mays
DAGSWIQDSAVMLSVSKEVPWYLVTNCRCYYAVIFFYFF

SEQ ID NO: 296 - protein, 58 aa, Zea mays
SHRQRNRIRASRRDQVFRVRLQAVLTIPSSSRTHRHWVFRSLRTHFTRLGATGAAAGG

SEQ ID NO: 297 - protein, 35 aa, Zea mays
YEYAFQIWFPIGCLALEMLVSVLYTCIKLVTSQIF

SEQ ID NO: 298 - protein, 29 aa, Zea mays
RALGALFHWMPESIRGREETTPRRINQDR

SEQ ID NO: 299 - protein, 43 aa, Zea mays
LGHRAGSPGHKGPGTTQVRRTPEVTAMRKNVTHRPKRRANTRT

SEQ ID NO: 300 - protein, 25 aa, Zea mays
MWISFSKHFYPNTTPLIYSIPSSPF

SEQ ID NO: 301 - protein, 57 aa, Zea mays
WNFKFEREDKRKGNKIEKKKKKERETTHWANSSVAGPTNLLSPQPALPPALTLGPHW

SEQ ID NO: 302 - protein, 39 aa, Zea mays
ERQELRLVEGLRGPSPPGDLCEWWWWSESREEGTDVSSR

SEQ ID NO: 303 - protein, 36 aa, Zea mays
HVRRRRRSTRARGGAASSRATDGRSQIQRRDDGDFP

SEQ ID NO: 304 - protein, 40 aa, Zea mays
KLGQTSWGKLELKPNKMSPKGEKREKGEGKDFESTKGKRP

SEQ ID NO: 305 - protein, 60 aa, Zea mays
ISKLTFLCHYLIDMGIKVEGETYIPGYFDMEDSSVNYNGNVLPYHKENKPSVFLTEKFTI

SEQ ID NO: 306 - protein, 39 aa, Zea mays
QPSPFLSPFTPARPNNISWSQPASGRRHHDPTSQPRPSR

SEQ ID NO: 307 - protein, 27 aa, Zea mays
IFFILFLYIYMFTFLDNLLSGVDVPLG

SEQ ID NO: 308 - protein, 31 aa, Zea mays
RRAVSSLAGIRFPFPVVVCCCYSPDTGAFPP

SEQ ID NO: 309 - protein, 36 aa, Zea mays
WKSEKNCGLSLLHNEKNQKLRHIIGILFSLLCLFPV

SEQ ID NO: 310 - protein, 62 aa, Zea mays
TVQKSEVIAPKSEHTGLSGVPLDCPVPQEDKGLQRSTAPNPNGRLTWHTPNNEQCHVQYTTG

SEQ ID NO: 311 - protein, 23 aa, Zea mays
GSRVFVPQIYKIKTILGRWNFSW

SEQ ID NO: 312 - protein, 55 aa, Zea mays
HVLHNRYSTDFGKNEMEVLKHAANYLEPAESEQLLELLASTQLKVMYQNYDWSIN

SEQ ID NO: 313 - protein, 43 aa, Zea mays
EEEEEEEEEEEEEEEEEEGNRVRLPACHRLRKKPAPRDAAAGR

SEQ ID NO: 314 - protein, 53 aa, Zea mays
TVVCQDKGPCYGKKVACPKRCFWSYSRSGSGYGAGGGGGSCTVDCKDKCTATC

SEQ ID NO: 315 - protein, 43 aa, Zea mays
VFEGTRPVADEKVKGTATEQRQNEGGTPRSSDAQANGRDKRRP

SEQ ID NO: 316 - protein, 34 aa, Zea mays
VPMPERDSIHRAGGSPWGVGLLLLLLLVMVSYQS

SEQ ID NO: 317 - protein, 127 aa, Zea mays
PAASSSSSVRCTSSRRDDAEFGCNGGGGRIVDEGMVVLRRRIHEMEAAERGWDPPEEWAAWEKEWYATYDADVCQQLVGVLQAFLVNSRPGVGVGLVAVLMLAVPASAFVLVSLLLDASRAVVSNLH